(19) United States
(12) Patent Application Publication
(10) Pub. No.: US 2022/0375544 A1
(43) Pub. Date: Nov. 24, 2022

(54) KIT AND METHOD OF USING KIT

(71) Applicant: CONGENICA LTD., Cambridge (GB)

(72) Inventors: Nick Lench, Cambridge (GB); Suzanne Drury, Cambridge (GB); Yogen Patel, Cambridge (GB); Tim Rayner, Cambridge (GB)

(21) Appl. No.: 17/761,419

(22) PCT Filed: Sep. 18, 2020
(86) PCT No.: PCT/GB2020/052266

§ 371 Date: Mar. 17, 2022

(30) Foreign Application Priority Data
Sep. 20, 2019 (GB) 1913639.9

(51) Int. Cl. G16B 30/00; C12Q 1/6883; G16B 20/10; G16B 20/20; G16B 40/20

(52) U.S. Cl. CPC G16B 30/00; C12Q 1/6883; G16B 20/10; G16B 20/20; G16B 40/20; C12Q 1/6869

(57) ABSTRACT

A kit for use in an apparatus for a genetic screening, where the kit when in operation, performs a wet-lab assay. The assay includes processing genetic material that is derived from one or more cell exomes, and detecting single nucleotide variants (SNVs), indels and copy number variations (CNVs) in genetic DNA readout from genetic material. The kit is executable as single assay that processes the genetic material. The kit includes a software product that is executable on a computing hardware to cause the computing hardware to invoke algorithms to process the genetic DNA readout by comparing portions of the genetic DNA readout against DNA sequence transcripts, to determine a probability of occurrence of the DNA sequence transcripts in the DNA readout data. The algorithms are used to detect both SNVs and CNVs concurrently in the genetic DNA readout from the genetic material and annotate clinically relevant CNVs.

### TECHNICAL FIELD

The present disclosure generally relates to genomics or systems, apparatus and processes for clinical genomics; more specifically, the present disclosure relates to kits or a method for (of) using the kits to perform a wet-lab assay for processing genetic material in order to identify accurately and cost-effectively multiple variant types in a single assay with significantly improved accuracy and efficiency The present disclosure further relates to systems and methods that efficiently acquire and accurately process genomic sequence datasets and address the effects of biases for accurate detection of copy number variants in a given genomic sequence dataset.

### BACKGROUND

With recent advancements in medical and computational technology, there has been rapid progress in respect of genomic sequencing and analysis of corresponding sequencing data. The sequencing data is commonly generated in short-read sequences, for example, between 50 and 300 deoxyribonucleic acid (DNA) bases, with these read sequences being distributed stochastically across an individual's genome. The genetic analysis involves a combination of many complex wet lab and in silico processes, wherein the processes start from acquiring a biological sample from a given individual to derive genetic material for further analysis. Contemporary sequencing technologies, for example next generation sequencing (NGS), are capable of sequencing long DNA molecules by converting them into smaller fragment molecules, sequencing the fragment molecules in amplified form to generate corresponding fragment sequences, and then piecing together the fragment sequences to generate a DNA read of the long DNA molecules. In certain scenarios, a genomic technique for sequencing the protein-coding region of genes in a genome (known as the exome) is used. Alternatively, a whole-genome sequencing approach may be used instead of exome sequencing but is expensive to implement as compared to exome sequencing approach. There are substantial differences in the biases and data errors introduced between whole-genome sequencing and exome sequencing, and further differences between each of the exome sequencing assays currently available, which makes the identification of different mutation types even more problematic.

Furthermore, such sequencing technologies, for example, NGS provides input data (e.g. exome sequence data) that forms a basis for identifying different mutation types (i.e. different types of variants) in the genome, which may or may not be responsible for the occurrence of ailments or abnormalities manifested as one or more phenotypes in the given individual. Examples of such different mutation types or variants present in the genome include, but are not limited to, single nucleotide variants (SNVs), copy number variations (CNVs), and indels. The SNVs occur in the genome when a single DNA base within the genome is substituted with a different DNA base. Detection of such SNVs may be performed easily as only one defaulter base pair needs to be identified, and is therefore well-known and researched in the art. On the other hand, the CNVs occur in the genome when a sequence of the DNA base pairs is duplicated or deleted in the genome. Generally, the size of CNVs may vary from a few dozen bases up to several mega-bases of the genome. Thus, detection of such CNVs is a complex task, as compared to SNVs.

Currently, there are many technical problems encountered in the processing of genetic material in order to identify different variant types. The disconnected approach to detection, visualization, and analysis of different variant types (SNVs, CNVs, and the like) using separate tests, tools, and platforms, the high costs involved in performing multiple tests in order to identify different mutation types, and the risk of missing certain variants when separate tests are conducted, are some of the technical problems encountered in the processing of genetic material in order to identify different mutation types. Typically, chromosomal microarrays have been the established standard for cytogenetics applications with detection of larger variant types, such as CNVs, whereas the NGS has been reserved usually for smaller mutation types, such as SNVs or mutations resulting from few base variations. With decreasing costs of an NGS assay, many systems and methods are being developed to obtain CNVs from sequencing data, as it is estimated that CNVs account for about 10-15% of the pathogenicity in rare diseases. Currently, different tests are required to be executed separately to detect different mutation types, i.e. SNVs, CNVs, and the like. In a recent study, it is estimated that microarray analysis detects only about 12% of causative events in patients with genetic disorders. Those patients without a causative finding are then referred to a second test which in most cases is DNA sequencing. Thus, performing two tests results in higher costs as well as longer time to assessment as to whether disease exists or not. Furthermore, it has been estimated that, about 5% of samples have multiple pathogenic variants, about 12% of samples have dual variants, i.e. include a combination of CNVs and SNVs. Such cases would be missed if exome sequencing or CNV analysis was performed alone at a given time point.

Furthermore, now-a-days, the decreasing costs have made it much more affordable to perform NGS sequencing and the need for deriving CNVs from NGS data has been rising. There are several tools available for CNV detection, but such tools are not user-friendly, and require user expertise (i.e. bioinformatics expertise). For example, most of the existing tools and systems are operable only using a command line (i.e. a text interface where commands to the program in the form of successive lines of text to interact with the computer), which is not easy-to-use. Furthermore, each tool is adept in only one domain. For example, some tools and systems are adept at being used for somatic or constitutional samples, while some are adept in the analysis of data from whole genome sequencing (WGS) but equally not suited for exome sequencing data. Moreover, some tools and systems are adept in genetic analysis using data from targeted gene panels to detect certain mutation types (i.e. variants) with clinically acceptable sensitivity and specificity. Moreover, a significant number of pathogenic genomic alterations fall between the gap of detection of such mutation types from NGS and microarray. Clinically, additional testing for such mutation types (variants) relies on Multiplex Ligation-dependent Probe Amplification (commonly known as MLPA), which is expensive and requires one kit per gene to be performed. In addition, this potentially increases the testing time. Furthermore, conventional assays do not allow an integrated approach to data analysis, visualization, and variant interpretation resulting in misinterpretation or missing out variants (i.e. due to low sequence coverage). Thus, it is a technical problem as conventional solutions require different tests to be performed, are difficult to implement in a clinical lab, and work as separate and disconnected solutions, i.e. separate determination of different mutation types, where the results are disconnected with each other, which results in inefficient downstream processing further resulting in comparatively low coverage (i.e. are suited only for specific domain area), and offer poor visualization of results.

Another challenge encountered is sample tracking. Maintaining sample integrity is paramount in the interpretation of variants. For example, samples undergo numerous physical steps from DNA extraction from a given sample to generation of sequencing data making it a vulnerable process leading to mixing up of samples. In addition, sample mix up can introduce clinical risk, delay provisioning of results, and further potentially leads to wastage of time and reagents, which has an adverse financial implication.

Additionally, pharmacogenomics is the study of how the genetic make-up of an individual can affect an individual's response to drugs, which can provide important information in trying to individualize drug selection and drug dosing to avoid adverse drug reactions, side effects and maximize drug efficacy. For example, the Food and Drug Administration (FDA) now includes pharmacogenomics information on the labels of more than 100 medications used across nearly every medical discipline, emphasizing its wide reach and potential impact of implementation. This genetic variation in an individual can affect how rapidly a given drug is activated or cleared from the human body and the amount of the given drug that may be required to elicit the desired target response. It is estimated that only 30-70% of patients respond positively to drugs, and patients may even face a potential risk of suffering an adverse drug reaction (ADR). Currently, widespread adoption of pharmacogenomic markers is largely limited to predesigned, targeted assays, meaning anyone undergoing exome sequencing would need a separate assay to be run, which requires more sample and incurs additional testing pathways and costs. Moreover, many standard NGS pipelines do not routinely call homozygous wildtype (due to the additional storage and computation requirement and also many of these variants are common in the population, and are therefore filtered out by standard filtering approaches), which is not desirable. Furthermore, in certain scenarios, many pathogenic mutations lie outside of the coding regions captured by some existing off-the-shelf exome assays. This can result in incorrect decision support for a physician to take precautionary measures, or treatment due to a missed assessment of a disease as a result of the causative mutation not being captured and sequenced in the off-the-shelf exome assays-based kits.

Therefore, in light of the foregoing discussion, there exists a need to overcome the aforementioned drawbacks associated with conventional kits, systems, and methods for processing of genetic material, analysis of genomic sequence data, and identification of multiple mutation types.

As with recent advancements in medical and computational technology, rapid progress in respect of genomic sequencing, analysis of corresponding sequencing data as such, the commonly generated sequencing data in short-read sequences, for example, between 50 and 300 deoxyribonucleic acid (DNA) bases, are distributed stochastically across a patient's genome. Such short-read sequencing data are produced using many different laboratory techniques, all of which introduce their own data errors or biases into the generated data, which is not desirable.

In certain scenarios, in order to reduce cost, the genomic regions sequenced are usually confined to a panel of genes which are known to be involved in pathogenesis, in a process known as “clinical exome sequencing”. The panel of genes is defined as a list of target regions within the genome, and typically within this contain a select set of genes or gene regions that have known or suspected associations with the disease or phenotype under study. Many capture assay kits are available, which are generally tailored to slightly different gene panels, and which use alternative designs and process to capture the sequences of interest. Alternatively, a whole-genome sequencing approach may be used instead of exome sequencing but is expensive to implement as compared to exome sequencing approach. There are substantial differences in the biases and data errors introduced between whole-genome sequencing and exome sequencing, and further differences between each of the exome sequencing assays currently available.

Further, such sequencing technologies provides input data that forms a basis for identification of several genetic variants or mutations in the genome, which may or may not be responsible for the occurrence of ailments or abnormalities manifested as phenotype in the given individual. Examples of such genetic variants or mutations present in the genome, include but are not limited to single nucleotide variants (SNVs), copy number variants (CNVs), and structural variants (SVs). A human DNA typically comprises DNA bases known as nucleotides, namely Adenine (A), Guanine (G), Cytosine (C) and Thymine (T) in pairs such that ‘A’ pairs with ‘T’ (A—T) and ‘C’ pairs with ‘G’ (C—G). The SNVs occur in the genome when a single DNA base within the genome is substituted with a different DNA base. For example, if ‘A’ is replaced with ‘G’, the original base pair that is A—T is replaced as a base pair G—T. In such a case, abnormalities arise in a genome of the individual due to the faulty base pair G—T. However, detection of such SNVs may be performed easily as only one defaulter base pair needs to be identified, and is therefore well-known and researched in the art. On the other hand, the CNVs occur in the genome when a sequence of the DNA base pairs is duplicated or deleted in the genome. Generally, the size of CNVs may vary from a few dozen bases up to several mega-bases of the genome. Thus, detection of such CNVs is a complex task, and not many existing systems and methods are able to identify the CNVs in the genome efficiently, and even if some CNVs are identified, there are many false positives, and certain other CNVs are missed. Moreover, the biases (or data errors) introduced in the generated short-read sequencing data, the biases introduced between whole-genome sequencing and exome sequencing, and further differences between each of the exome sequencing assays currently available, make the CNV calling (i.e. detection) process even more problematic. Furthermore, there are certain known applications used to detect copy number variants. However, as a result of aforesaid problems of biases (or data errors) and also due to the use of multiple different sequencing assay types, such applications vary in their performance, and are thus not reliable and accurate.

Therefore, in light of the foregoing discussion, there exists a need to overcome the aforementioned drawbacks associated with conventional systems and methods for processing and analysis of genomic sequence data.

### SUMMARY

The present disclosure seeks to provide an improved kit for use in an apparatus, where the kit is used for genetic screening and performs a wet-lab assay, which includes processing genetic material that is derived from one or more cell exomes, and detecting single nucleotide variants (SNVs), indels and copy number variations (CNVs) in a genetic DNA readout from the genetic material. The present disclosure also seeks to provide a method for (of) using a kit, which performs a wet-lab assay that includes processing genetic material that is derived from one or more cell exomes, and detecting SNVs, indels, and CNVs in a genetic DNA readout from the genetic material. The present disclosure seeks to provide a solution to an existing problem of low coverage representative of misinterpretation of variants or missing out variants in genomic sequencing readout data derived from one or more cell exomes. The present disclosure further seeks to provide a solution to an existing problem of a disconnected approach to detection, visualization, and/or further analysis different variant types (SNVs, CNVs, and indels) using separate tests, tools, and platforms, and high costs involved in performing multiple tests in order to identify different variant types.

An aim of the present disclosure is to provide a solution that overcomes at least partially the problems encountered in prior art, and provides an improved kit, and method that provides an integrated solution that is user-friendly, cost-effective, and is able to detect different variant types (SNVs, CNVs, and indels) concurrently from a single assay with comparatively high coverage resulting in significantly low probability in missing out variants, and further allows visualization and further analysis of detected different variant types in a connected and integrated approach.

In one aspect, the present disclosure provides a kit for use in an apparatus and for a genetic screening, wherein the kit, when in operation, performs a wet-lab assay, wherein the assay includes processing genetic material that is derived from one or more cell exomes, wherein the assay detects single nucleotide variants (SNVs), indels and copy number variations (CNVs) in genetic DNA readout from the genetic material,

characterized in that
the kit is executable as a single assay that processes the genetic material; and
the kit includes a software product that is executable on a computing hardware to cause the computing hardware to invoke one or more algorithms to process the genetic DNA readout by comparing portions of the genetic DNA readout against one or more DNA sequence transcripts, to determine an occurrence of variants corresponding to the one or more DNA sequence transcripts in the DNA readout data,
wherein the one or more algorithms include:
(i) an algorithm for detecting SNVs, indels and CNVs concurrently in the genetic DNA readout from the genetic material in the single assay;
(ii) an algorithm for annotating clinically relevant CNVs present in the genetic DNA readout from the genetic material;
(iii) an algorithm that prioritizes one or more portions of the genetic DNA readout from the genetic material depending on phenotype associated with the one or more portions
(iv) an algorithm that detects variant calling for pharmacogenomic (PGx) markers and
(V) an algorithm configured to sample tracking SNPs in the single assay.

In another aspect, the present disclosure provides a method for (of) using a kit, wherein the kit, when in use, performs a wet-lab assay, wherein the assay includes processing genetic material that is derived from one or more cell exomes, wherein the assay detects single nucleotide variants (SNVs), indels and copy number variations (CNVs) in genetic DNA readout from the genetic material, characterized in that the method includes:

(i) applying the kit as a single assay that processes the genetic material; and
(ii) executing a software product of the kit on computing hardware to cause the computing hardware to invoke one or more algorithms to process the genetic DNA readout by comparing portions of the genetic DNA readout against one or more DNA sequence transcripts, to determine an occurrence of variants corresponding to the one or more DNA sequence transcripts in the DNA readout data,
wherein the one or more algorithms include:
(a) an algorithm for detecting SNVs, indels and CNVs concurrently in the genetic DNA readout from the genetic material in the single assay;
(b) an algorithm for annotating clinically relevant CNVs present in the genetic DNA readout from the genetic material;
(c) an algorithm that prioritizes one or more portions of the genetic DNA readout from the genetic material depending on phenotype associated with the one or more portions; and
(d) an algorithm that detects variant calling for pharmacogenomic (PGx) markers; and
(e) an algorithm configured to sample tracking SNPs in the single assay.

Embodiments of the present disclosure substantially eliminate, or at least partially address, the aforementioned problems in the prior art, and enables the kit to be executed as a single assay that processes the genetic material so that the different variant types (SNVs, CNVs, indels, and PGx markers) are determined cost-effectively from the single assay and at the same time have a high coverage resulting in significantly low probability in missing out variants. The present disclosure also addresses the problem of a disconnected approach by providing an integrated solution that enables not only detection, but also concurrent visualization and further analysis of different variant types in a connected, user-friendly, and integrated approach, which reduces risk of misinterpretation of genetic variants.

The present disclosure also seeks to provide an improved system that acquires and processes genomic sequence datasets to detect copy number variants. The present disclosure also seeks to provide an improved method for (of) acquiring and processing genomic sequence datasets to detect copy number variants. The present disclosure seeks to provide a solution to an existing problem of inefficient and unreliable detection of copy number variants in a given genomic sequence dataset due to the biases in the given genomic sequence dataset. Moreover, the present disclosure further seeks to address an existing problem of how to identify an efficient and the best application from multiple different applications for a specific genomic sequence dataset that helps in accurate and reliable detection of the copy number variants in the specific genomic sequence dataset, which potentially have biases (or data errors).

An aim of the present disclosure is to provide a solution that overcomes at least partially the problems encountered in prior art, and provides an improved system and method that addresses the effects of the biases for efficient and accurate detection of copy number variants in a given genomic sequence dataset by identification of an optimal application that is reliable and efficient for the given genomic sequence dataset.

In one aspect, the present disclosure provides a system that acquires and processes genomic sequence dataset to detect one or more copy number variants (CNVs) therein, the system comprising:
an apparatus configured to process at least a portion of a genome of a subject to generate a raw genomic sequence dataset; and
a computing arrangement comprising a data memory device and control circuitry, wherein the control circuitry is configured to:
   acquire the raw genomic sequence dataset from the apparatus and a plurality of candidate CNV detection applications prestored in the data memory device;
   execute a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth;
   combine the baseline CNVs obtained from each of the plurality of candidate CNV detection applications to generate a set of baseline CNVs;
   generate a simulated genomic sequence dataset by simulation of a set of artificial CNVs in at least one target region of the raw genomic sequence dataset by use of a simulation application prestored in the data memory device, wherein the simulated genomic sequence dataset comprises the set of artificial CNVs and the set of baseline CNVs;
   record a location of each artificial CNV of the set of artificial CNVs and each baseline CNV of the set of baseline CNVs in the simulated genomic sequence dataset;
   execute a second CNV calling in the simulated genomic sequence dataset by use of each of the plurality of candidate CNV detection applications;
   eliminate the set of baseline CNVs from CNVs obtained from the second CNV calling in the simulated genomic sequence dataset to obtain a set of new CNVs;
   determine a location of each new CNV of the set of new CNVs in the simulated genomic sequence dataset based on the recorded location of the set of artificial CNVs;
   determine a degree of recall and a degree of precision associated with each of the plurality of candidate CNV detection applications based on a comparison of the location of the set of new CNVs with the location of the set of artificial CNVs;
   select one of the plurality of candidate CNV detection applications as being optimal, based on a combination of the degree of recall and the degree of precision for calling the copy number variants in genomic sequence data; and
   utilize the selected candidate CNV detection application for calling of CNVs in the genomic sequence data.

In another aspect, an embodiment of the present disclosure provides a system that processes a raw genomic sequence dataset to detect one or more copy number variants (CNVs) therein, the system comprising:
a computing arrangement comprising a data memory device and control circuitry, wherein the control circuitry is configured to:
acquire the raw genomic sequence dataset and a plurality of candidate CNV detection applications prestored in the data memory device;
execute a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth;
combine the baseline CNVs obtained from each of the plurality of candidate CNV detection applications to generate a set of baseline CNVs;
generate a simulated genomic sequence dataset by simulation of a set of artificial CNVs in at least one target region of the raw genomic sequence dataset by use of a simulation application prestored in the data memory device, wherein the simulated genomic sequence dataset comprises the set of artificial CNVs and the set of baseline CNVs;
record a location of each artificial CNV of the set of artificial CNVs and each baseline CNV of the set of baseline CNVs in the simulated genomic sequence dataset;
execute a second CNV calling in the simulated genomic sequence dataset by use of each of the plurality of candidate CNV detection applications;
eliminate the set of baseline CNVs from CNVs obtained from the second CNV calling in the simulated genomic sequence dataset to obtain a set of new CNVs;
determine a location of each new CNV of the set of new CNVs in the simulated genomic sequence dataset based on the recorded location of the set of artificial CNVs;
determine a degree of recall and a degree of precision associated with each of the plurality of candidate CNV detection applications based on a comparison of the location of the set of new CNVs with the location of the set of artificial CNVs;
select one of the plurality of candidate CNV detection applications as being optimal, based on a combination of the degree of recall and the degree of precision for calling the copy number variants in genomic sequence data; and
utilize the selected candidate CNV detection application for calling of CNVs in the genomic sequence data.

In yet another aspect, an embodiment of the present disclosure provides a method for (of) acquiring and processing genomic sequence datasets to detect one or more copy number variants (CNVs) therein, wherein the method is implemented using a system that comprises an apparatus and a computing arrangement, wherein the method comprises:
processing, by use of the apparatus, at least a portion of a genome of a subject to generate a raw genomic sequence dataset;
acquiring, by use of a control circuitry of the computing arrangement, the raw genomic sequence dataset from the apparatus and a plurality of candidate CNV detection applications prestored in a data memory device of the computing arrangement;
executing, by use of the control circuitry, a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth;
combining, by use of the control circuitry, the baseline CNVs obtained from each of the plurality of candidate CNV detection applications to generate a set of baseline CNVs;
generating, by use of the control circuitry, a simulated genomic sequence dataset by simulation of a set of artificial CNVs in at least one target region of the raw genomic sequence dataset by use of a simulation application prestored in the data memory device, wherein the simulated genomic sequence dataset comprises the set of artificial CNVs and the set of baseline CNVs;
recording, by use of the control circuitry, a location of each artificial CNV of the set of artificial CNVs and each baseline CNV of the set of baseline CNVs in the simulated genomic sequence dataset;
executing, by use of the control circuitry, a second CNV calling in the simulated genomic sequence dataset by use of each of the plurality of candidate CNV detection applications;
eliminating, by use of the control circuitry, the set of baseline CNVs from CNVs obtained from the second CNV calling in the simulated genomic sequence dataset to obtain a set of new CNVs;
determining, by use of the control circuitry, a location of each new CNV of the set of new CNVs in the simulated genomic sequence dataset based on the recorded location of the set of artificial CNVs;
determining, by use of the control circuitry, a degree of recall and a degree of precision associated with each of the plurality of candidate CNV detection applications based on a comparison of the location of the set of new CNVs with the location of the set of artificial CNVs;
selecting, by use of the control circuitry, one of the plurality of candidate CNV detection applications as being optimal, based on a combination of the degree of recall and the degree of precision for calling the copy number variants in genomic sequence data; and
utilizing, by use of the control circuitry, the selected candidate CNV detection application for calling of CNVs in the genomic sequence data.

In yet another aspect, an embodiment of the present disclosure provides a method for (of) acquiring and processing genomic sequence dataset to detect one or more copy number variants (CNVs) therein, wherein the method is implemented using a system that comprises a computing arrangement, wherein the method comprises:
acquiring, by use of a control circuitry of the computing arrangement, a raw genomic sequence dataset and a plurality of candidate CNV detection applications prestored in a data memory device of the computing arrangement;
executing, by use of the control circuitry, a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth;
combining, by use of the control circuitry, the baseline CNVs obtained from each of the plurality of candidate CNV detection applications to generate a set of baseline CNVs;
generating, by use of the control circuitry, a simulated genomic sequence dataset by simulation of a set of artificial CNVs in at least one target region of the raw genomic sequence dataset by use of a simulation application prestored in the data memory device, wherein the simulated genomic sequence dataset comprises the set of artificial CNVs and the set of baseline CNVs;
recording, by use of the control circuitry, a location of each artificial CNV of the set of artificial CNVs and each baseline CNV of the set of baseline CNVs in the simulated genomic sequence dataset;
executing, by use of the control circuitry, a second CNV calling in the simulated genomic sequence dataset by use of each of the plurality of candidate CNV detection applications;
eliminating, by use of the control circuitry, the set of baseline CNVs from CNVs obtained from the second CNV calling in the simulated genomic sequence dataset to obtain a set of new CNVs;
determining, by use of the control circuitry, a location of each new CNV of the set of new CNVs in the simulated genomic sequence dataset based on the recorded location of the set of artificial CNVs;
determining, by use of the control circuitry, a degree of recall and a degree of precision associated with each of the plurality of candidate CNV detection applications based on a comparison of the location of the set of new CNVs with the location of the set of artificial CNVs;
selecting, by use of the control circuitry, one of the plurality of candidate CNV detection applications as being optimal, based on a combination of the degree of recall and the degree of precision for calling the copy number variants in genomic sequence data; and
utilizing, by use of the control circuitry, the selected candidate CNV detection application for calling of CNVs in the genomic sequence data.

In yet another aspect, an embodiment of the present disclosure provides a computer program product comprising a non-transitory computer-readable storage medium having computer-readable instructions stored thereon, the computer-readable instructions being executable by a computerized device comprising processing hardware to execute the aforementioned method.

In yet another aspect, an embodiment of the present disclosure provides a method for (of) acquiring and processing genomic sequence dataset to detect one or more copy number variants (CNVs) therein, wherein the method is implemented using a system that comprises a computing arrangement, wherein the method comprises:
acquiring, by use of a control circuitry of the computing arrangement, a raw genomic sequence dataset and a plurality of candidate CNV detection applications prestored in a data memory device of the computing arrangement;
executing, by use of the control circuitry, a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth;
combining, by use of the control circuitry, the baseline CNVs obtained from each of the plurality of candidate CNV detection applications to generate a set of baseline CNVs;
generating, by use of the control circuitry, a simulated genomic sequence dataset by simulation of a set of artificial CNVs in at least one target region of the raw genomic sequence dataset by use of a simulation application prestored in the data memory device, wherein the simulated genomic sequence dataset comprises the set of artificial CNVs and the set of baseline CNVs;
recording, by use of the control circuitry, a location of each artificial CNV of the set of artificial CNVs and each baseline CNV of the set of baseline CNVs in the simulated genomic sequence dataset;
executing, by use of the control circuitry, a second CNV calling in the simulated genomic sequence dataset by use of each of the plurality of candidate CNV detection applications;
eliminating, by use of the control circuitry, the set of baseline CNVs from CNVs obtained from the second CNV calling in the simulated genomic sequence dataset to obtain a set of new CNVs;
determining, by use of the control circuitry, a location of each new CNV of the set of new CNVs in the simulated genomic sequence dataset based on the recorded location of the set of artificial CNVs;
determining, by use of the control circuitry, a degree of recall and a degree of precision associated with each of the plurality of candidate CNV detection applications based on a comparison of the location of the set of new CNVs with the location of the set of artificial CNVs;
selecting, by use of the control circuitry, one of the plurality of candidate CNV detection applications as being optimal, based on a combination of the degree of recall and the degree of precision for calling the copy number variants in genomic sequence data; and
utilizing, by use of the control circuitry, the selected candidate CNV detection application for calling of CNVs in the genomic sequence data.

Embodiments of the present disclosure substantially eliminate or at least partially address the aforementioned problems in the prior art, and enables selection of the optimal application for detection of the copy umber variants in the genomic sequence dataset. The selected optimal application for a specific genomic sequence dataset helps in accurate and reliable detection of the copy number variants in that genomic sequence dataset.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1A is a block diagram of a kit used in an apparatus, in accordance with an embodiment of the present disclosure;

FIG. 1B is a block diagram of a kit used in an apparatus, in accordance with another embodiment of the present disclosure;

FIG. 2 is an illustration of an exemplary scenario for implementation of a kit to perform a bespoke wet-lab exome assay, in accordance with an embodiment of the present disclosure;

FIG. 3 is a flowchart depicting steps of a method of using a kit that performs a wet-lab assay, in accordance with an embodiment of the present disclosure; and

FIG. 4 is a flowchart depicting steps of a method of using a kit that performs a wet-lab assay, in accordance with another embodiment of the present disclosure.

FIG. 5A is a block diagram of a system that acquires and processes genomic sequence dataset to detect copy number variants (CNVs), in accordance with an embodiment of the present disclosure;

FIG. 5B is an illustration of a network environment of a system that acquires and processes genomic sequence dataset to detect copy number variants (CNVs), in accordance with another embodiment of the present disclosure; and

FIGS. 6A and 6B is a flowchart depicting steps of a method for (of) acquiring and processing genomic sequence dataset to detect copy number variants (CNVs), in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, the present disclosure provides a kit for use in an apparatus, for a genetic screening, wherein the kit, when in operation, performs a wet-lab assay, wherein the assay includes processing genetic material that is derived from one or more cell exomes, wherein the assay detects single nucleotide variants (SNVs), indels and copy number variations (CNVs) in genetic DNA readout from the genetic material,

characterized in that
the kit is executable as a single assay that processes the genetic material; and
the kit includes a software product that is executable on a computing hardware to cause the computing hardware to invoke one or more algorithms to process the genetic DNA readout by comparing portions of the genetic DNA readout against one or more DNA sequence transcripts, to determine an occurrence of variants corresponding to the one or more DNA sequence transcripts in the DNA readout data,
wherein the one or more algorithms include:
(i) an algorithm for detecting SNVs, indels and CNVs concurrently in the genetic DNA readout from the genetic material in the single assay;
(ii) an algorithm for annotating clinically relevant CNVs present in the genetic DNA readout from the genetic material;
(iii) an algorithm that prioritizes one or more portions of the genetic DNA readout from the genetic material depending on phenotype associated with the one or more portions;
(iv) an algorithm that detects variant calling for pharmacogenomic (PGx) markers; and separately sample tracking SNPs in the single assay.

In another aspect, an embodiment of the present disclosure provides a method for (of) using a kit, wherein the kit, when in use, performs a wet-lab assay, wherein the assay includes processing genetic material that is derived from one or more cell exomes, wherein the assay detects single nucleotide variants (SNVs), indels and copy number variations (CNVs) in genetic DNA readout from the genetic material, characterized in that the method includes:

(i) applying the kit as a single assay that processes the genetic material; and
(ii) executing a software product of the kit on computing hardware to cause the computing hardware to invoke one or more algorithms to process the genetic DNA readout by comparing portions of the genetic DNA readout against one or more DNA sequence transcripts, to determine an occurrence of variants corresponding to the one or more DNA sequence transcripts in the DNA readout data,
wherein the one or more algorithms include:
(a) an algorithm for detecting SNVs, indels and CNVs concurrently in the genetic DNA readout from the genetic material in the single assay;
(b) an algorithm for annotating clinically relevant CNVs present in the genetic DNA readout from the genetic material;
(c) an algorithm that prioritizes one or more portions of the genetic DNA readout from the genetic material depending on phenotype associated with the one or more portions; and
(d) an algorithm that detects variant calling for pharmacogenomic (PGx) markers and separately sample tracking SNPs in the single assay.

The present disclosure provides an integrated solution to detect, visualize, and further analyze different variant types (i.e. a combination of SNVs, CNVs, and indels) concurrently from a single assay performed using the aforementioned kit and method. The disclosed kit is executable as a single assay that processes a genetic material, such as an exome or targeted gene (i.e. exome) panel panels, to obtain the genetic DNA readout from the genetic material. The kit is used for genetic screening. Examples of the genetic screening include, but are not limited to a preconception screening, a preimplantation genetic screening, or an application related to assisted reproduction technology. The different variant types (i.e. SNVs, CNVs, indels, and PGx markers) are detected together in a single assay in a connected and integrated approach, which significantly increases coverage in terms of genetic variants detection and reduces misinterpretation of variants and avoids any inadvertent missing out of potential variants (i.e. different variant types that are clinically relevant) in the genetic DNA readout derived from cell exome. The kit utilizes the software product and extensive dataset having DNA sequence transcripts to determine an occurrence of variants corresponding to the one or more DNA sequence transcripts in the DNA readout data, which effectively handles and reduces the effect of biases, if any in exome sequencing, and provides a capability to the kit to detect multiple (i.e. dual, triple, and more) pathogenic variants (i.e. combination of CNVs and SNVs or CNVs, SNVs, and PGx markers) directly from extracted samples. The kit allows visualization and further analysis of detected different variant types in a connected and integrated approach.

Throughout this application, variant or genetic variation is referred to or can be seen in the context of an individual of any species, groups or population, and is observed in genes as well as in alleles. Facts that cause genetic variation may include but are not limited to gene mutations, crossing over, recombination, genetic drift, gene flow, and environmental factors or intensify the process of natural selection. Variants may bring evolutionary changes.

Further, the terms single nucleotide variant (SNV) and single nucleotide polymorphism (SNP) are used equivalently herein.

The aforementioned kit does not require to run multiple assays and tests, and is thus highly cost-effective. Furthermore, the kit prevents sample mix up, thereby improving clinical safety, preventing wastage of time and reagents, and thus providing savings in terms of time and cost. The kit that is used in the apparatus can be operated using a graphical user interface, which is easy-to-use, and the entire kit and method are easy to implement in a clinical lab. The kit executes the software product on computing hardware to cause the computing hardware to invoke one or multiple algorithms in a systematic manner to process the genetic DNA readout, which ensures a coherent analysis of different variant types; the computing hardware can be a contemporary laptop computer, computing workstations or similar (for example, a contemporary quad-core processor computer whose processors are operating at circa 3 GHz). The kit also enables the calling of homozygous wildtype, via the underlying algorithm(s), to identify a presence of variants therein without filtering out such variants to further reduce the chance of missing out any variant of clinical use. The kit can be easily designed as a bespoke clinical exome assay specialized for an entity to be more effective in accordance with the application area of the entity. For example, causative variants that manifest into a phenotype (e.g. a disease) are effectively captured in the bespoke clinical exome assay performed by the kit. Alternatively stated, the kit enables detection, visualization, and analysis of multiple variant types that cause rare diseases in an individual, which is currently overlooked due to a disconnected approach in processing of genetic material derived from one or more cell exomes, and analysis of the genetic DNA readout obtained from such processed genetic material.

The present disclosure provides a kit for use in an apparatus. The kit, when in operation, performs a wet-lab assay, wherein the assay includes processing genetic material that is derived from one or more cell exomes, and wherein the assay detects single nucleotide variants (SNVs), indels and copy number variations (CNVs) in genetic deoxyribonucleic acid (DNA) readout from the genetic material. The “kit” herein refers to an exome capture kit. Specifically, the kit is a single assay exome capture kit for detecting multiple variant types. The kit includes components that enable processing genetic material that is derived from at least an exome, and a software product upon which the components are configured to operate; the components optionally include, for example, pre-prepared plate arrays, for example. The term “apparatus” refers to a machine or a system of which the kit is a part or in which the kit operates in association with the apparatus. In an example, the apparatus may be deoxyribonucleic acid (DNA) readout apparatus, such as a sequencing platform. The sequencing platform may be a large-scale sequencer or a compact benchtop sequencer. The kit, when in use in the apparatus, is configured to perform the wet-lab assay to obtain the genetic DNA readout. The term “cell exome” refers to a complete sequence of one or more exons in protein-coding genes in the genome of the subject. According to an embodiment, the cell exome is exome plus (exome+). The exome plus refers to protein-coding exons as well as non-coding regions with known contributions to pathogenesis (e.g. known splice-modifying sites and/or transcription factor binding sites). The sequences of the one or more exons in the gene are transcribed, such that the exons remain within the mRNA, whereas introns (non-coding regions of the gene) are removed by mRNA splicing and contribute to the final protein product encoded by that gene. The kit in use with the apparatus is configured to process a target region, such as the cell exome to derive the genetic material. The identification of the variants, such as the SNVs, the indels, and the CNVs in the cell exome of the subject may provide information about the genetic disorders and the genetic diseases that the subject may possess.

According to an embodiment, the kit is operated in a plurality of stages. Specifically, the plurality of stages refers to four sequential stages, such as a first selection stage, a second wet-lab stage, a third data processing stage, and a fourth visualization stage, which works in synchronization with each other in a connected and integrated approach. The first selection stage refers to a selection stage in which an entity that uses a kit is able to select a set of features-of-interest from a plurality of features as per customized requirements (i.e. the kit operates as a bespoke clinical exome assay configurable as per a requirement for a particular vendor, entity, or an end-user). The second wet-lab stage refers to genetic material processing stage using the kit in accordance with the selected set of features-of-interest in the first selection stage to obtain the genetic DNA readout from the genetic material. The third data processing stage refers to data processing pipeline stage in which the output (i.e. the genetic DNA readout data) from the second data processing stage is processed in accordance with a selected set of features-of-interest in the first selection stage. The fourth visualization stage refers to a visualization stage in which a graphical user interface is rendered for visualization and further analysis of the processed data at the third data processing stage.

In the first selection stage, a user (on the purchase or optionally after the purchase of the kit), is provided with options to choose features as per requirement. The kit allows data processing, variant filtering, variant prioritization, and visualization of processed data (e.g. reports). The data processing features and visualization features are configurable and are made available to the owner of the kit as per requirement. In an implementation, a token provides access to (or activates) certain selected features. Examples of the plurality of features that the kit allows being selected as per choice include, but are not limited to exome sequencing preferences and a plurality of custom variants identification modules. Such plurality of features are configurable using the kit. In an example, using the kit, an end-user is allowed to select whole exome sequencing (WES), a shallow whole-genome sequencing (sWGS), or a combination thereof (i.e. WES±sWGS or sWGS±WES), and an exome plus analysis feature. WES and sWGS use next generation sequencing (NGS) to identify genetic variants in the coding regions (exons) of genes, encompassing disease-causing variants. The term “exome plus” refers to protein-coding exons as well as non-coding regions with known contributions to pathogenesis (e.g. known splice-modifying sites and/or transcription factor binding sites). The exome plus thus is a more powerful tool to identify different types of variants having clinical and pharmacogenomic use (e.g. protein-truncating variants).

In addition to the exome sequencing preferences, following features are selectable (i.e. can be opted-in or opted-out) as per choice: i) a prenatal module; ii) early-infantile epileptic encephalopathy (EIEE) neuro-medical module; and a carrier screening panel module. The prenatal module includes a combination of curated and known DNA sequence transcripts dataset to identify variants in prenatal testing. For example, the prenatal module includes at least 2598 fetal anomalies gene transcripts. The EIEE neuro-medical module includes a combination of curated and known DNA sequence transcripts dataset to identify variants related to EIEE. For example, the EIEE neuro-medical module includes at least 5019 epilepsy gene Havana transcript features. The EIEE is a rare neurological disorder characterized by seizures. The EIEE is a severely progressive syndrome, has an early onset (e.g. usually before the age of one), and some children with EIEE potentially go on to develop other epileptic disorders later in life. It is observed that epilepsy, in a significant percentage of children, is wrongly identified and treated as gastrointestinal disorders. There are more than 300 genes known to cause EIEE, and thus the neuro-medical module provides comparatively more extensive and comprehensive coverage require coverage to such genes (e.g. in comparison to conventional panels include only subsets of these genes). The carrier screening panel module attempts to identify a subject (or a couple) at elevated risk of having a child affected with one or preselected set of Mendelian conditions, thereby enabling consideration of alternative productive options and early intervention strategies. Optionally, an expanded carrier screening (ECS) panel module is used, which identifies reproductive risks for multiple (e.g. greater than 10) diseases.

In the second wet-lab stage, the kit allows a DNA sample to be extracted locally for sequencing purposes. The extraction of DNA sample from a biological subject is performed using known methods of DNA/RNA isolation. The basic criteria that any method of DNA isolation (i.e. extraction) from any biological sample type should meet include: efficient extraction, sufficient amount of DNA/RNA extracted for downstream processes, such as next generation sequencing (NGS), removal of contaminants, and quality and purity of DNA. In an example, ultraviolet absorbance is usually used to assess the purity of the extracted DNA. For a pure DNA sample, the ratio of absorbance at 260 nm and absorbance at 280 nm is about 1.8. The biological sample of a subject refers to a laboratory specimen taken, preferably non-invasively, by sampling under controlled environments, that is, gathered matter of a medical subject's tissue, fluid, or other material derived from the subject. Examples of the biological sample include, but are not limited to, blood, throat swabs, sputum, surgical drain fluids, tissue biopsies, amniotic fluid, or sample of the fetus.

According to an embodiment, the DNA sample is sheared. The shearing is an enzymatic shearing (e.g. using a restriction enzyme) or an acoustic shearing. It is to be understood by a person of ordinary skill in the art that any other DNA fragmentation method can be used (such as nebulization or the long DNA molecule is potentially fragmented chemically or using a transposable element), without limiting the scope of the disclosure. The fragmented DNA samples after shearing are used to prepare sWGS (shallow-low-level) library that incorporate unique molecular identifiers (UMI) and an index of a corresponding sample (i.e. a sample index) in case the sWGS feature is selected in the sequence preferences. Furthermore, the fragmented DNA samples after shearing are also used in WES library preparation that also incorporate UMI and sample index in case the WES feature is selected in the sequence preferences in the first selection stage. In WES, protein-coding regions of the genome are targeted and enriched via specific hybridization of genomic fragments with complementary oligonucleotides, or ‘baits’. These targeted regions are then sequenced using high throughput next-generation sequencing (NGS) technologies. Thereafter, the sWGS and the WES libraries are pooled (i.e. combining sWGS and the WES libraries) for high-coverage paired-end exome sequencing (which enables full exome plus downstream analysis). The sequencing of such selected libraries is performed. In an example, the sequencing is performed using a defined number of base pairs (bp) paired end reads (short reads) (e.g. using NGS sequencing). In another example, the sequencing is performed with long-read sequencing (i.e. with the capacity to sequence on average over 10 kb in a single read).

In an example, in NGS, in some cases where the length of DNA sections is relatively longer, for example longer than 250 base pairs, the fragments are ligated with generic adaptors (i.e. small piece of known DNA located at the read extremities) and annealed to a glass slide using the adaptors (e.g. in Illumina based sequencing). In some cases, mRNA transcripts are isolated, which correspond to the coding regions of functional genes, for example in exome sequencing. Such mRNA transcripts are subjected to reverse-transcription to obtain cDNA fragments. According to an embodiment, the kit in use with the apparatus is further configured to execute sequencing of the plurality of complementary deoxyribonucleic acid (cDNA) fragment molecules concurrently in a next generation sequencing (NGS) process to generate the genetic material to obtain the genetic DNA readout. Notably, sequencing, for example, DNA sequencing, is the process of determining the sequence of nucleotides in a given section of DNA. In NSG, the sequencing is done in a parallel manner using sequencing-by-synthesis, to produce a set of concurrent data, composed of millions of short sequencing reads. A computing device is then employed to detect a base at each read location site in each image, which is then used to construct a sequence. The readout of the sequence by the apparatus corresponds to the genetic DNA readout data (i.e. sequencing data).

According to an embodiment, the kit does not require setting up thousands of PCR reactions. The kit allows enrichment of the exome plus regions in a single assay (e.g. a single solution test tube). Targeted exome plus sequencing allows for parallel enrichment of target regions in one simple step for assessment of potential disease-associated regions, and candidate genes. The sequencing data obtained from the sequencing is uploaded to a cloud-based sequence analysis and visualization platform. In an example, the sequencing data (i.e. the genetic DNA readout data) is uploaded in the form of binary base call (BCL), FASTQ, Binary Alignment Map (BAM), Variant Call Format (VCF), or Browser Extensible Data (BED) format. The kit is communicatively coupled to the cloud-based sequence analysis and visualization platform.

In an example, the raw genomic sequencing readout refers to binary base call (BCL) data, i.e. raw sequencing readout directly from a sequencing machine. The FASTQ format is a text-based format for storing base call and corresponding quality information. The BAM format is a compressed binary version of a sequence alignment format (SAM) file that is used to represent aligned sequences. The VCF format is a text file used for storing gene sequence variations (variations of a gene). The BED format provides a flexible way to define the data lines that are displayed in an annotation track. The sequencing data is uploaded using the selected token(s) that provide access to selected modules (i.e. features) in the first selection stage. Optionally, a sample tracking assay of choice (i.e. as per selection performed in the first selection stage) is also run locally. In such a case, the output of the sample tracking assay performed previously is also uploaded in the cloud-based sequence analysis and visualization platform. In an example, the output of the sample tracking assay includes SNP data that is used as markers to avoid sample mix-ups.

According to an embodiment, the third data processing stage, i.e. the data processing pipeline stage begins with the upload of the genetic DNA readout data (i.e. the sequencing data). A specific processing pipeline(s) is triggered in accordance with the selected features (e.g. module token) in the first selection stage. In an example, an initial alignment of the sequencing data is performed with reference genomic dataset. The sequencing data is aligned to, for example, the GRCh38/hg38 human genome build assembly. In an example, it is checked that all of the reads had a quality score above threshold (e.g. greater than 10) at every position. This reduces the number of error-prone reads, thereby improving alignment results. By use of the alignment data or raw sequencing data, sample tracking SNPs with quality control are generated. The SNPs and in some cases short tandem repeat markers are potentially used for genetic sample tracking to avoid sample mix-ups. Further, UMI deduplication is susceptible to being performed on the sequencing data (i.e. raw sequencing data uploaded or the alignment data. The DNA fragments of a long DNA molecule, incorporates an identifier, known as a unique molecule identifier (UMI), prior to amplification thereof. Notably, a UMI is a random sequence of nucleotides that is in a range of 8 to 16 base pairs long. During amplification, a given UMI corresponding to a given fragment molecule is attached to each of duplicate molecules generated from the given fragment molecule. During sequencing, the UMI is read as a separate piece of read data. UMI deduplication is performed on the sequencing data (i.e. raw sequencing data uploaded or the alignment data obtained from the initial alignment of the sequencing data performed with reference genomic dataset). As the result of the demultiplexing, the UMI sequences (or other barcodes if any), are segregated from the actual sequencing data of each DNA fragment molecules (i.e. the set of forward reads and the set of reverse reads).

Moreover, the kit is executable as a single assay that processes the genetic material. The kit typically performs the single wet-lab assay to process the genetic material in order to obtain the genetic DNA readout, which in turn is used detect the SNVs, the indels and the CNVs in the genetic DNA readout. The single assay itself is able to detect the SNVs, the indels and the CNVs in the genetic DNA readout from the genetic material. It will be appreciated that the SNVs occur in the cell exome when a single DNA base within the cell exomes is substituted with a different DNA base. For example, if “A” is replaced with “G”, the original base pair that is “A—T” is replaced as a base pair “G—T”. In such a case, abnormalities arise in exome of the subject due to the faulty base pair “G—T”. The SNVs may contribute to several types of genetic disorders or diseases such as sickle-cell anemia, β-thalassemia, cystic fibrosis and so forth. Notably, the severity of illness in the subject and the way the subject responds to treatments are also manifestations of genetic variations, such as SNVs. For example, a single-base variant in an apolipoprotein E (APOE) gene is associated with a lower risk for Alzheimer's disease; it will be appreciated that UMI deduplication refers to a process where non-biological duplicates are removed when processing genetic readout data. Furthermore, the “indels” refer to small genetic variations or variants associated with insertion or deletion of bases, such as A, T, C or G in the genome of the subject. In an example, the indels may vary from 1 base pair to 10,000 base pairs in length, including insertion and deletion events that may be separated by many years, and may not be related to each other in any way. Notably, the indels may further include microindels, such that a microindel corresponds to an indel that results in a change of 1 to 50 base pairs in length. The indels may also contribute to several types of genetic disorders or diseases such as Bloom syndrome that is a rare autosomal recessive disorder characterized by short stature of the subject, predisposition to the development of cancer and genomic instability. Notably, Bloom syndrome is predominantly observed in Jewish and Japanese populations. Thus, for processing of the genetic DNA readout of a Jewish or a Japanese subject, the target region may include the genes responsible for Bloom syndrome. Moreover, the CNVs refers to sections of the genome of the subject that are repeated and the number of repeats in the genome varies between subjects in the human population. The CNV is a result of copy number variation event, which is a type of duplication or deletion event that affects a considerable number of base pairs. Typically, differences in the DNA sequence in genomes contribute to the uniqueness of the subject. These differences potentially influence most traits, including susceptibility to disease. Since CNVs often encompass genes, the detection of CNVs have important roles both in human disease and drug response. Moreover, in comparison to other genetic variants (e.g. SNPs and indels), CNVs are larger in size and can often involve complex repetitive DNA sequences. In certain cases, CNVs also encompass entire genes, which have a specific protein encoding function ascribed to them. For these reasons, CNVs are potentially more amenable to misinterpretation, and are difficult to detect as compared to other genetic variants. It will be appreciated that the CNVs are linked with genetic disorders, such as genetic diseases and the like. In human genome, currently most CNVs are found to be benign variants that do not directly cause disease. However, there are several instances where CNVs affect critical developmental genes and cause rare diseases, for example intellectual disability. There are certain reports of CNVs causing neurological disorders affecting the nervous system and contributing to Parkinson's Disease and Alzheimer's Disease as well as neuropsychiatric disorders such as bipolar disorder and schizophrenia. There could be thousands more CNVs in the human population, which lie undetected due to various reasons and problems discussed above. Thus, the kit in use with apparatus is configured to process the genetic DNA readout to detect the SNVs, the indels and the CNVs therein. Subsequently, the accurate and comprehensive detection of the SNVs, the indels and the CNVs finds applications in decision support and facilitates to pinpoint a target region in the cell exome of the genome that needs to be focused for treatment of the identified rare genetic disorder due to a specific detected SNV, indel or CNV, for example, by performing gene therapy. In some cases, certain SNVs, indels or CNVs could be employed to add discrimination power in forensics.

Furthermore, the kit includes a software product that is executable on a computing hardware to cause the computing hardware to invoke one or more algorithms to process the genetic DNA readout by comparing portions of the genetic DNA readout against one or more DNA sequence transcripts, to determine an occurrence of variants corresponding to the one or more DNA sequence transcripts in the DNA readout data. The term “software product” refers to any collection or set of instructions executable by a computer or other digital system, such as a computing hardware so as to configure the computing hardware to perform a task that is the intent of the software product. Additionally, the software product is intended to encompass such instructions stored in storage medium such as random-access memory (RAM), a hard disk, optical disk, or so forth, and is also intended to encompass so-called “firmware” that is a software stored on a ROM or so forth. Optionally, the software product refers to software application and associated data. Such software product is organized in various ways, for example the software product includes software components organized as libraries, Internet-based programs stored on a remote server or so forth, source code, interpretive code, object code, directly executable code, and so forth. It will be appreciated that the software product optionally invokes system-level code or calls to other software residing on a server or other location to perform certain functions, such as to instruct a computing hardware. The term “computing hardware” refers to a computational element that is operable to respond to and process instructions that drives the kit in use with the apparatus. Optionally, the computing hardware includes, but is not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the term “computing hardware” optionally refers to one or more individual hardware, processing devices and various elements associated with a computing device that are optionally shared by other computing devices. Additionally, the one or more individual computing devices and elements are arranged in various architectures for responding to and processing the instructions that drive the kit, when in use with the apparatus. The computing hardware is configured to invoke the one or more algorithms, that, for example, are stored in the computing hardware as one or more applications. The term “algorithm” refers to a set of instructions required to perform a specific task. Herein, the one or more algorithms are invoked (namely, executed) by the computing hardware to perform tasks, such as a determination of occurrence of variants corresponding to the one or more DNA sequence transcripts in the DNA readout data. The one or more algorithms are invoked to process the genetic DNA readout by comparing portions of the genetic DNA readout against the one or more DNA sequence transcripts. Such processing of the genetic DNA readout is required to determine the occurrence of variants corresponding to the one or more DNA sequence transcripts in the DNA readout data. Examples of the one or more algorithms include, but are not limited to regression-based algorithms, read depth data-based algorithms, and the like.

The term “DNA sequence transcripts” refers to reference genomic sequences, such as gene variant sequences derived from publicly-available DNA databases or self-curated DNA databases comprising verified information about disease causing variants present in the sequences. Such DNA sequence transcripts are used as a reference for comparison of the DNA readout data to determine the occurrence of variants corresponding to the one or more DNA sequence transcripts in the DNA readout data.

According to an embodiment, the one or more DNA sequence transcripts include consensus coding sequence (CCDS) transcripts. The CCDS transcripts are a dataset of the protein-coding regions (i.e. exome) that are identically annotated on human and mouse reference genome assembly in genome annotations. Identically annotated coding regions, that are generated using an automated pipeline process and pass multiple quality assurance checks, are assigned a stable and tracked identifier (CCDS ID). Additionally, the CCDS transcripts dataset is maintained through stringent quality assurance testing and manual curation. A sequence alignment of the genetic DNA readout against CCDS transcripts sequences identifies any potential regions that are different. The chances of having different types of variants in those regions is prominent. In an example, the sequence alignment is performed using an alignment tool (e.g. offline or online version of Basic Local Alignment Search Tool (BLAST) or other alignment tools. Further, sequence alignment of the genetic DNA readout (i.e. a query sequence) with other more DNA sequence transcripts (i.e. target sequences) provides a thorough understanding of specific types of variants and corresponding disease-causing phenotypes. An alignment score is typically generated in each alignment of query and target sequence using a sequence coverage and a sequence similarity. A cent percent sequence coverage and sequence similarity indicate an identical sequence (i.e. a perfect match), which in turn represents that the subject has the genetic variant responsible for a disease with confirmation. Furthermore, analysis using the GUI rendered on a display screen associated with the apparatus, is performed to check whether the genetic variant is dominant of recessive, or how likely the genetic variant will result in a phenotype arising.

According to an embodiment, the one or more DNA sequence transcripts include at least one morbid gene RefSeq transcript. The morbid gene RefSeq transcript is a gene sequence acquired from a publicly-available database (known as morbid gene RefSeq transcript database) comprising comprehensive collection of genes and genetic phenotypes. Notably, the morbid gene RefSeq transcript database is a publicly available database, and is maintained by a collaboration between the National Library of Medicine and William H. Welch Medical Library at Johns Hopkins, USA, and is regularly updated. The morbid gene RefSeq transcript includes information about known Mendelian disorders, such as sickle-cell anemia, Tay-Sachs disease, cystic fibrosis, xeroderma pigmentosa and the like. The morbid gene RefSeq transcript comprise information of at least 15,000 genes in its database. Typically, the morbid gene RefSeq transcript is focused on establishing a relationship between a genotype and a phenotype. According to an embodiment, the one or more DNA sequence transcripts include at least 4091 morbid gene RefSeq transcripts. The morbid gene RefSeq transcript database comprise at least 4091 morbid gene RefSeq transcripts that provides information about the human genes and the genetic phenotypes. In case an alignment score (above a specified threshold) is generated by a sequence alignment of the genetic DNA readout against the morbid gene RefSeq transcripts, it indicates that a portion of the DNA readout has a variant responsible for a specific Mendelian disorder.

According to an embodiment, the one or more DNA sequence transcripts include at least one fetal anomaly gene transcript. The fetal anomaly gene transcript is a gene variant sequence acquired from a database that comprises information about the variants present in the human genome that are responsible for the fetal anomalies. The fetal anomaly refers to genetic defects that develops in the fetus that potentially affect pregnancy, complicate delivery process for a woman and potentially pose serious threat to the life of a child. Notably, the fetal anomalies, also known as birth defects, include structural changes that potentially develop due to genetic defects in one or more parts of the fetus's body that potentially increase the chance of morbidity and mortality of the child. Furthermore, the fetal anomalies potentially cause deficiencies that potentially deteriorate a health of the child, hamper the development and lower the quality of life of the child. According to an embodiment, the one or more DNA sequence transcripts include at least 2598 fetal anomalies gene transcripts. The fetal anomaly gene transcript database comprises at least 2598 fetal anomalies gene transcripts that provides information about the genes causing defects such as amniotic band syndrome, achondroplasia, Down syndrome, Turner's syndrome, spinal dysraphism, conjoined twins, polyhydramnios, Rh incompatibility, gastrointestinal atresia, and so forth. The kit is configured to retrieve any updated fetal anomalies gene transcripts data from the database so that only latest variant data is used in sequence alignment and analysis. In case an alignment score (above a specified threshold) is generated by a sequence alignment of the genetic DNA readout against the fetal anomaly gene transcripts, it indicates that a portion of the DNA readout has a variant responsible for a specific fetal anomaly.

According to an embodiment, the one or more DNA sequence transcripts include at least one epilepsy anomaly gene transcript. The epilepsy anomaly transcript is a gene variant sequence acquired from a database that comprises information related to epilepsy, more specifically early infantile epileptic encephalopathy (EIEE) in children. The causes of the EIEE are potentially genetic, such as due to specific type of variants in the genome of the child. The epilepsy anomaly transcript is used as a reference to identify presence of such variants that potentially cause an onset of EIEE in the child. The identification of the variants that potentially cause EIEE are optionally used for disease assessment purposes for a fetus. Typically, the EIEE is an age-related disorder that is characterized by an onset of tonic spasms within the first three months of life of the child, independent of the sleep cycle, that can occur over hundreds of times per day, consequently leading to psychomotor impairment and death of the child. Thus, such epilepsy anomaly transcript aids in providing information related to EIEE, that is potentially useful to detect specific gene variants responsible for EIEE in the fetus for prenatal screening.

According to an embodiment, the one or more DNA sequence transcripts include at least 5019 epilepsy gene Havana transcript features. The Havana (Human and Vertebrate Analysis and Annotation) transcripts emphasize on areas such as alternatively spliced transcripts and pseudogenes. The Havana transcript annotation takes into account and utilize various data, such as CpG islands (i.e. a short sequence of DNA in which the “C—G” sequence has a frequency higher than other sequences), gene predictions, repeats and genome signatures. Furthermore, the annotation software used by Havana transcript features is Distributed Annotation System (DAS) aware, thus the HAVANA transcript is able to link to external data sources. In case an alignment score (above a specified threshold) is generated by a sequence alignment of the genetic DNA readout against the epilepsy gene Havana transcript sequences, it indicates that a portion of the DNA readout has a variant responsible for a specific epilepsy disorder.

According to an embodiment, the one or more DNA sequence transcripts include at least one ACMG 59 gene RefSeq transcript. The ACMG i.e. American College of Medical Genetics and Genomics 59 gene RefSeq transcript is a database that comprise information about 59 genes at present. The database comprises a list of genes that are reported as incidental findings or secondary findings. The aim of creating the ACMG 59 gene RefSeq transcript is the identification and management of risks for selected highly penetrant genetic disorders through established interventions that are aimed at preventing or significantly reducing morbidity and mortality in a human.

According to an embodiment, the one or more DNA sequence transcripts include likely pathogenic variants and non-coding variants of DNA sequence (ClinVar). ClinVar is a publicly-available database that comprise information about relationships among medically important variants and phenotypes. The ClinVar database includes information that reports human variation, interpretations of the relationship of that variation to human health and the evidence supporting each interpretation. Notably, each record in the ClinVar database represents a submitter, a variation and a phenotype. The ClinVar database may represent the interpretation of a single allele, compound heterozygotes, haplotypes and combinations of alleles in different genes as well. It will be appreciated that a majority of a portion of a human genome is non-coding DNA, thus, information about the non-coding variants in such non-coding DNA may also be present in the ClinVar database. In case an alignment score (above a specified threshold) is generated by a sequence alignment of the genetic DNA readout against the pathogenic variants and non-coding variants of DNA sequence, it indicates that a portion of the DNA readout has a variant responsible for a specific disorder as indicated by corresponding annotation of the variant in the Clinvar database.

According to an embodiment, the one or more DNA sequence transcripts include at least one sample-tracking SNPs. The biological samples undergo numerous physical steps from DNA extraction through generation of sequencing data, thereby making them vulnerable to inaccurate processing, for example, by mix-up of the biological samples. The identification of positive results is done using orthologous methods, however the identification of negative results is difficult using such orthologous methods. Additionally, the biological sample mix up can delay a return of the results, wastes time and reagents which has a financial implication. Thus, the one or more DNA sequence transcripts include at least one sample-tracking SNPs, that aids in tracking of the biological sample throughout the process, thereby reducing chances of mix-up.

Moreover, the one or more algorithms include an algorithm for detecting both SNVs and CNVs, and optionally indels, concurrently in the genetic DNA readout from the genetic material in the single assay. The software product that is executable on the computing hardware causes the computing hardware to invoke the algorithm to perform detection of both SNVs and CNVs concurrently as dual variants in the genetic DNA readout from the genetic material. The detection of SNVs and CNVs in the genetic DNA readout enables identification of genetic diseases or disorders that may appear in the subject due to a combination of any of the detected SNVs and CNVs. Notably, the SNVs and the CNVs coexist throughout the genome of the subject, thus, the SNVs influence genotype measurement of the CNVs and vice-versa. In an embodiment, the combination of SNVs and the CNVs are detected as dual variants in a same genomic region. The data generated during SNV genotyping can be used for extraction of information, such as locations of CNVs in the genetic DNA readout. Furthermore, some CNVs may be detected by using a number of common SNV arrays. The algorithm is configured to detect the SNVs and the CNVs in the genetic DNA readout to identify effects of the combinations of various SNVs and the CNVs concurrently on the subject.

Furthermore, the one or more algorithms include an algorithm for annotating clinically relevant CNVs present in the genetic DNA readout from the genetic material. The CNVs detected in the exome region of the genetic DNA readout are typically of clinical relevance. The CNVs present in the exome region of the genetic DNA readout of the subject have a greater probability of contributing towards pathogenesis than the CNVs present in the intron regions. Thus, the CNVs present in the exome region are assumed to be of clinical relevance as they may be linked to the occurrence of the genetic disorders and the genetic diseases in the subject. The algorithm is configured to annotate the clinically relevant CNVs out of all the CNVs detected in the genetic DNA readout of the subject. Moreover, it may be required to identify a specific type of CNV that is responsible for occurrence of a particular genetic disorder. In such a case, the algorithm is configured to detect and annotate that specific type of CNV that is of clinical relevance. In an example, a clinical study requires identification of a neurological disorder named “Huntington's disease”. The algorithm is then configured to detect tri-nucleotide repeat of the “CAG” base pairs in the Huntingtin gene. The repetition of the “CAG” tri-nucleotide more than 36 times generally indicates that the Huntington's disease is likely to develop. Thus, the algorithm annotates the repetitions of the “CAG” tri-nucleotide out of all the CNVs detected in the genetic DNA readout to verify if the Huntington's disease is likely to develop in the subject.

Moreover, the one or more algorithms include an algorithm that prioritizes one or more portions of the genetic DNA readout from the genetic material depending on phenotype associated with the one or more portions. The variants in a portion of the genetic DNA readout are potentially responsible for occurrence of a specific phenotype in the subject. The algorithm is configured to prioritize such one or more portions of the genetic DNA readout, for the identification of variants that potentially contribute towards specific phenotypes of interest. In an example, phenotypes associated with a subject are: upward slanting shape of eyes, white spots on the iris of the eyes, a flat nasal bridge, a protruding tongue, a single flexion furrow of the fifth finger and so forth. The one or more portions of the genetic DNA readout that are associated with the abovementioned phenotypes are prioritized over other portions of the genetic DNA readout. Such prioritization enables easy and faster detection of genetic abnormalities, as the results are confined to specific variants that may have caused the phenotypes and are of clinical relevance. The algorithm is able to identify the genetic disorders, syndromes or diseases that may be linked with the abovementioned phenotypes.

Furthermore, the one or more algorithms include an algorithm that detects variant calling for pharmacogenomic (PGx) markers and separately sample tracking SNPs. The PGx markers helps in the determination of a relationship between the various variants present in the genome of the subject and the effect of medicines on the subject due to the various variants. It will be appreciated that each subject may experience a different reaction from a medicine, due to the difference in the variants present in each subject. Thus, pharmacogenomics helps in establishing a relationship between the variants and the medicines, in order to provide personalised and better diagnosis to each subject depending on the variants present in the genome of the subject. For example, an enzyme CYP2D6 is encoded in the human body by a gene “CYP2D6”. The efficiency and the amount of enzyme CYP2D6 produced between different humans vary considerably, depending upon the presence, absence, copies, and the like of the gene “CYP2D6” in the humans. Some humans are able to eliminate certain drugs that are metabolized by the enzyme CYP2D6 quickly, whereas some humans eliminate the drugs metabolized by the enzyme CYP2D6 slowly. It will be appreciated that, quick metabolization of the drug results in reduced efficacy of the drug, whereas slow metabolization of the drug may result in toxicity. Thus, dosage of such drugs needs to be administered and personalized for each human accordingly. The algorithm is configured to detect variant calling, such as for the gene “CYP2D6” for pharmacogenomic (PGx) markers.

According to an embodiment, the software product includes an algorithm that, when executed on the computing hardware, detects at least one of duplications and deletions in the DNA readout data relative of the DNA sequence transcripts, and wherein the genetic screening for which the kit is used includes at least one of a preconception screening, a preimplantation genetic screening, or an application related to assisted reproduction technology, and wherein the genetic material is processed using single cell sequencing. The duplications and deletions, such as indels are detected by the algorithm to identify the genetic disorders or the genetic diseases associated with them. For example, cystic fibrosis, Bloom syndrome and so forth are caused due to indels present in the genetic DNA readout. It is known that different disease-causing variant types have different ranges in terms of lengths. For example, SNPs affect single bases and indels usually affect fewer than ten bases, but deletions and duplications span hundreds to thousands of bases. Thus, unlike SNPs and indels, which are typically much shorter than NGS short reads (obtained by sequencing), and thus are clearly visible and identifiable within single DNA read, whereas the deletions and duplications that exceed an NGS read length, require proper analysis from NGS sequencing data. Thus, the duplication and deletion variants are detected based on comparison with the DNA sequence transcripts. In an implementation, probes are potentially used. Probes that successfully bind to genomic DNA are competent for amplification, thus the amount of amplified probe is proportional to the amount of genomic DNA (i.e. a deletion that halves the amount of genomic DNA will yield half as much amplified probe, thereby indicating deletion). Similarly, a duplication increases (doubles) the amount of genomic DNA at particular site and will yield double as much amplified probe in same time as compared to other amplified probes.

According to an embodiment, the kit is used for a preconception screening, a preimplantation genetic screening, or an application related to assisted reproduction technology. The preconception screening refers to a genetic screening that allows to determine whether a given individual (parent) is at risk of conceiving a child with a genetic disorder. The preimplantation genetic screening refers to a genetic screening that allows to determine genetic defects in embryos created through in vitro fertilization (IVF) before pregnancy. Typically, in preimplantation genetic screening, embryos from presumed chromosomally normal genetic parents are screened for aneuploidy. The assisted reproduction technology related to technologies and procedures to help in achieving pregnancy. The genetic material is processed using single cell sequencing, which provides sequencing data (e.g. exome or transcriptome) from individual cell with NGS technologies, providing a better understanding of the function or gene expression of an individual cell.

According to an embodiment, the kit is operated to detect the copy number variations (CNVs) in genetic DNA readout from the genetic material further comprises a control circuitry configured to: receive the genetic DNA readout and a plurality of candidate CNV detection applications; execute a first CNV calling to obtain baseline CNVs in randomly selected regions of the genetic DNA readout by use of each of the plurality of candidate CNV detection applications, wherein the baseline CNVs are pre-existent CNVs in the genetic DNA readout recognized as a ground truth; combine the baseline CNVs obtained from each of the plurality of candidate CNV detection applications to generate a set of baseline CNVs; generate a simulated genomic sequence dataset by simulation of a set of artificial CNVs in at least one target region of the genetic DNA readout by use of a simulation application, wherein the simulated genomic sequence dataset comprises the set of artificial CNVs and the set of baseline CNVs; record a location of each artificial CNV of the set of artificial CNVs and each baseline CNV of the set of baseline CNVs in the simulated genomic sequence dataset; execute a second CNV calling in the simulated genomic sequence dataset by use of each of the plurality of candidate CNV detection applications; eliminate the set of baseline CNVs from CNVs obtained from the second CNV calling in the simulated genomic sequence dataset to obtain a set of new CNVs; determine a location of each new CNV of the set of new CNVs in the simulated genomic sequence dataset based on the recorded location of the set of artificial CNVs; determine a degree of recall and a degree of precision associated with each of the plurality of candidate CNV detection applications based on a comparison of the location of the set of new CNVs with the location of the set of artificial CNVs; select one of the plurality of candidate CNV detection applications as being optimal, based on a combination of the degree of recall and the degree of precision for calling the copy number variants in genomic sequence data; and utilize the selected candidate CNV detection application for calling of CNVs in the genomic sequence data.

According to an embodiment, the control circuitry of the kit is further configured to determine the degree of recall associated with each of the plurality of candidate CNV detection applications by identification of: a true positive, if a location of a new CNV of the set of new CNVs and a corresponding location of an artificial CNV of the set of artificial CNVs match; a false positive, if a location of a new CNV of the set of new CNVs is detected at a location that is different than a location of an artificial CNV of the set of artificial CNVs; and a false negative, if no new CNV of the set of new CNVs is detected at a location an artificial CNV of the set of artificial CNVs.

According to an embodiment, the control circuitry of the kit is further configured to measure an extent of overlap of a location of a new CNV of the set of new CNVs with a corresponding location of an artificial CNV of the set of artificial CNVs, for determination of the degree of precision associated with each of the plurality of candidate CNV detection applications.

According to an embodiment, the control circuitry of the kit is configured to allocate a highest degree of precision to a first candidate CNV detection application among the plurality of candidate CNV detection applications, based on the measured extent of overlap of the location of the new CNV of the set of new CNVs with the corresponding location of the artificial CNV of the set of artificial CNVs, by use of each of the plurality of candidate CNV detection applications.

According to an embodiment, the control circuitry of the kit is further configured to set a specified threshold for determination of the extent of overlap of the location of the new CNV of the set of new CNVs with the corresponding location of the artificial CNV of the set of artificial CNVs.

According to an embodiment, the genetic DNA readout from the genetic material is generated by whole genome sequencing, an exome sequencing, or both.

According to an embodiment, the control circuitry of the kit is further configured to generate a precision-recall curve relationship associated with each of the plurality of candidate CNV detection applications, and wherein the selection of one of the plurality of candidate CNV detection applications as optimal depends upon a balance between the degree of recall and the degree of precision, wherein the balance between the degree of recall and the degree of precision related to each of the plurality of candidate CNV detection applications is indicated by a corresponding area-under-precision-recall-curve in the generated precision-recall curve relationship.

According to an embodiment, the kit further comprising a wet-laboratory configured to process a biological sample of the subject in the wet-laboratory arrangement to derive at least the portion of the genome of the subject to generate the genetic DNA readout.

According to an embodiment, the software product includes an algorithm that, when executed on the computing hardware, detects one or more intergenic variants present in the DNA readout data relative of the DNA sequence transcripts. Some pathogenic variants caused by the variants lie outside of the coding regions captured by the exome assays. The failure of detection of the variants that lie outside the coding regions by the exome assays potentially results in missing out on identifying causative variant events, thereby cause misinterpretation of gene variants affected by such one or more intergenic variants. For example, gene regulatory elements, like cis or trans elements are usually conserved, but if changed due to variant results in failure to bind corresponding transcription factors. This in turn results in failure of gene transcription and formation of protein. The failure to produce a protein can potentially result in a disorder. Hence, in order to avoid misinterpretation or missing out of any gene variant, the intergenic variants present in the DNA readout data are also detected based on sequence alignment with related DNA sequence transcripts. If an identical match (or above a specified similarity threshold (e.g. 90% similarity)) is found for an intergenic variant, it is confirmed that the subject has a particular intergenic variant.

According to an embodiment, the software product includes an algorithm that, when executed on the computing hardware, detects heteroplasmic variants to recognize the most functionally important mitochondrial variants that contribute to phenotype (e.g. a disease) among a huge number of candidates. The mtDNA data is extracted from the sequencing data (i.e. from sWGS and WES data). In an example, “MToolBox” tool is used, which is an automated pipeline for heteroplasmy annotation and prioritization analysis of human mitochondrial variants in high-throughput sequencing, known in the art. In an example, reads mapped on mtDNA are realigned onto the nuclear genome (GRCh38/hg38), to discard nuclear mitochondrial sequences and amplification artifacts.

According to an embodiment, the software product includes an algorithm that, when executed on the computing hardware, provides a visualization arrangement implemented using a graphical user interface (GUI) to communicate visually results of detection of both SNVs and CNVs in the genetic DNA readout, annotation of clinically relevant CNVs present in the genetic DNA readout, prioritization of one or more portions of the genetic DNA readout from the genetic material depending on phenotype associated with the one or more portions and detection of variant calling for pharmacogenomic (PGx) markers and sample tracking SNPs. The visualization arrangement refers to a collection of one or more components that are used for visual representation of the results. In an example, the visualization arrangement is a laptop computer, a personal computer, medical monitors, and the like. The “GUI” refers to a structured set of user interface elements rendered on a visualization arrangement, such as a display screen. Optionally, the GUI rendered on the visualization arrangement is generated by any collection or set of instructions executable by an associated digital system. Additionally, the GUI is operable to interact with the user to convey graphical and/or textual information and receive input from the user. Furthermore, the GUI elements refer to visual objects that have a size and position in the GUI. A user interface element may be visible, though there may be times when a user interface element is hidden. A user interface control is considered to be a user interface element. Text blocks, labels, text boxes, list boxes, lines, and images windows, dialog boxes, frames, panels, menus, buttons, icons, etc. are examples of user interface elements. In addition to size and position, a user interface element may have other properties, such as a margin, spacing, or the like. The algorithm is configured to communicate to the GUI to visually represent the detected variants, annotation of the clinically relevant CNVs present in the genetic DNA readout, prioritization of one or more portions of the genetic DNA readout from the genetic material depending on phenotype associated with the one or more portions and detection of variant calling for pharmacogenomic (PGx) markers and sample tracking SNPs. According to another embodiment, the algorithm is configured to communicate to the GUI to visually represent the duplications and deletions in the DNA readout data relative of the DNA sequence transcripts, intergenic variants present in the DNA readout data relative of the DNA sequence transcripts, and combined SNV and CNV filtering and interpretation by a mode of genetic inheritance.

According to an embodiment, in the fourth visualization stage of the plurality of stages, the GUI is rendered to communicate and interact with results of detection in the third data processing pipeline stage based on a plurality of defined settings. The plurality of defined settings (hereinafter referred to as preset settings), knowledgebase, and panels are selected and applied via the rendered GUI (i.e. the visual interface) in an interactive manner. In other words, the results of various data processing operations are rendered on the GUI for further analysis. Furthermore, the data processing is performed based on the preset settings, knowledgebase, and panels selected and applied via the rendered visual interface. The third data processing stage and the fourth visualization stage are executed in synchronization to each other. In an implementation, a first preset setting of the plurality of preset settings (preset 1) allows to preload primary gene panel(s) and associated data (e.g. the aforesaid prenatal module or the aforesaid EIEE module panel). In a case where no identifiable pathogenic variant is detected by the primary panels based on predefined rules, a second preset setting (preset 2) of the plurality of preset settings is applied. In the second preset setting, Mendelian inheritance (e.g. OMIM or MORBID) data, and HPO data are preloaded and rendered alongside the preload primary gene panel(s) and associated data.

According to an embodiment, the software product includes algorithm that, when executed on the computing hardware, provides a combined SNV and CNV filtering and interpretation by a mode of genetic inheritance, wherein the mode of genetic inheritance includes a potential for recessive genes being present. The mode of genetic inheritance (also simply referred to as mode of inheritance (MOI)) refers to a manner by which a genetic trait or a genetic disorder is passed from one generation to a next generation. For example, the mode of inheritance may be autosomal dominant mode of genetic inheritance, autosomal recessive mode of genetic inheritance, X-linked dominant mode of genetic inheritance, X-linked recessive mode of genetic inheritance, multifactorial mode of genetic inheritance, mitochondrial inheritance mode of genetic inheritance and the like. The combined SNV and CNV filtering process is optionally performed by, for example, using the mode of genetic inheritance. In an example, a person has a carrier gene related to color blindness, i.e. the person is not color blind but carries a recessive gene for color blindness. The variants in the genome of the person are filtered out to identify the presence of the carrier gene related to color blindness. Such identification helps to identify a probability of occurrence of color blindness in the offspring of the person. At least one dominant carrier gene is required in parent to manifest into a phenotype, and thus the filtering helps to avoid any misinterpretation related to probability of the offspring developing a phenotype. The combined SNV and CNV filtering process optionally also comprises, for example, selection of the confident variants that are recognized to be present in the genetic DNA readout, and elimination of the variants that potentially have been falsely identified. Such filtering enables accurate detection of the variants in the genetic DNA readout. Furthermore, the filtering of the SNV and CNV is optionally performed to extract a subset of variants, combine the variants from several exome assays, and so forth. In contrast to existing analysis approaches, which are disconnected with wet-lab processing and visualization, and operate with the use of separate systems, and devices, and sometime even operating entities (e.g. laboratories, clinics, research centers), the disclosed kit is designed as a bespoke clinical exome assay specialized for an entity to be more effective in accordance with application area of the entity, and enables not only detection, but further visualization, and further analysis of multiple variant types including dual variants or triple variants concurrently using the single assay that cause rare diseases in an individual, which is currently overlooked (e.g. overlooking of the dual variants CNV and SNV in a same genomic region) due to a disconnected approach in processing of genetic material derived from one or more cell exomes, and separate or disconnected analysis of the genetic DNA readout obtained from such processed genetic material. Since the disclosed kit allows detection of both SNVs and CNVs (i.e. combination of the SNVs and CNVs) concurrently as dual variants in the genetic DNA readout from the genetic material in the single assay, thus the capability of having the combined SNV and CNV filtering and interpretation is provided by the kit in an integrated manner, where the clinical significance of such dual variants is discernible easily at least by use of the combined SNV and CNV filtering and interpretation. Further, such filtering allows to identify a probability of occurrence of a clinically significant (or relevant) phenotype (e.g. a genetic disorder) in the offspring of a person, which has practical implication in the preconception screening, the preimplantation genetic screening, and/or applications related to assisted reproduction technology.

According to an embodiment, the determination of the occurrence of variants in the DNA readout data further comprises detecting short tandem repeats (STR) and VNTR (variable number tandem repeats) in the genetic DNA readout data. The STR is typically, a unit of 1 to 13 base pairs repeated several times in a row on the DNA strand. Optionally, 1 to 6 repeated base pairs form the STR. Notably, the STR are hyper-mutable sequences in the human genome. The STR are detected in the genetic DNA readout that are utilized in various applications such as forensics, population genetics and so forth. The VNTR may be found in intergenic regions as well as in both the noncoding and coding regions of a variety of different genes. The diseases caused due to the long and highly polymorphic tandem repeats are the repeat expansion diseases. The tandem repeats in the coding sequence of the genome may result in the generation of toxic or malfunctioning proteins, whereas the tandem repeats in the noncoding regions may cause generation of chromosome fragility, silencing of the genes in which they are located, modulation of transcription and translation, sequestering of proteins involved in processes such as splicing and cell architecture, and so forth.

The determination of the occurrence of variants in the DNA readout data further comprises detecting mosaic variants in the genetic DNA readout data. Mosaicism refers to presence of two or more populations of cells with genetic differences found within one organism (such as the subject) and is often due to the acquisition of somatic variants during development. Typically, somatic variants are common in cancerous cells. In an implementation, “MuTect” tool is used to identify mosaic variants. In an example, a cohort of parent/affected child trios data is potentially used in such mosaic variant detection that are low-frequency variants, as compared to other types of variants.

According to an embodiment, the different variants called (duplication and deletion variants including further CNV calling, SNV, indel, STR, and VNTR, are tagged as per the type of variant at corresponding site on the genetic DNA readout data. The tagging (or annotations) is performed for the variants that meet gene mode of inheritance (MOI) (i.e. observed gene MOI) with expected MOI in a family. Mode of inheritance (MOI) is a manner in which a genetic trait or disorder is passed from one generation to the next. For example, autosomal dominant, autosomal recessive, X-linked dominant, X-linked recessive, multifactorial, and mitochondrial inheritance are genetic trait or disorder passed from one generation to the next. Each mode of inheritance results in a characteristic pattern of affected and unaffected family members due to various combination of recessive-dominant alleles.

According to an embodiment, the software product, when executed on the computing hardware, determines whether a variant is an inherited variant or a de novo variant. The variant passed-on in an offspring by one of its parents is referred to as inherited variant, whereas a genetic variation that is present for the first time in the offspring as a result of a variant in a germ cell (egg or sperm) of one of the parents, or a variant that arises in the fertilized egg itself during early embryogenesis is referred to as de novo variant. The de novo variants may contribute to a number of severe early-onset genetic disorders, such as intellectual disability, autism spectrum disorder, developmental diseases and the like. Thus, in the third data processing pipeline stage, the detected variant is determined as whether it is inherited variant or a de novo variant, as the effect of both the variants vary in the individuals.

According to an embodiment, the detected variants are categorized on primary gene panel(s) (i.e. variant tiering is performed). Furthermore, variant prioritization is performed for all the detected variants based on genes-of-interest. Moreover, an evidence code is auto populated when the detected variants match with prestored variant sequences acquired from a specified data source that defines gene variations and corresponding disorders. For example, ACMG evidence code is auto populated in case the detected variants match with the ACMG provided variant sequences. The ACMG stands for the American College of Medical Genetics and Genomics that has published recommendations for reporting incidental findings in the exons of certain genes (typically 59 genes are prescribed). For example, the recent version recommendation is ACMG SF v2.0 (available at PubMed 27854360), which indicates comprehensive list of variations of each gene and corresponding disorders with clinical significance (e.g. likely pathogenic) and associated data. As discussed above, the results of various data processing operations executed at the third data processing stage are rendered on the GUI (i.e. the visual interface) for further analysis, and also the data processing is performed based on the preset settings, knowledgebase, and panels selected and applied via the rendered visual interface. Thus, in addition to first and second preset setting, a third preset setting is selectable via the GUI. The third preset setting is panel agnostic and is used for configuration of a report template that can be used for decision support for assessment of a disease(s). For example, the carrier screening panel report with Bayes carrier risk calculated is rendered on the visual interface. Bayes carrier risk refers to a probability of a subject having a child affected with one or preselected set of Mendelian conditions. The Bayes carrier risk is calculated using Bayes theorem in which when a given number of predefined conditions are met, it is calculated a probability score depending on how many conditions are actually met from a total number of given conditions. The more the number of conditions is met, the more is the probability of the subject having the risk of passing on the disease to child (i.e. at high Bayes carrier risk). The Bayes theorem is implemented as conditions to be met using state tables that defines the conditions and checks how many are met at a given time to calculate the Bayes carrier risk.

According to an embodiment, other research preset options are selectable for visual analysis. A fourth preset setting of the plurality of defined settings is selectable via the GUI. The fourth preset setting allows cohort analysis and filtering to be performed based on shared alleles (e.g. variants that are shared and detected by multiple detections algorithms). A fifth preset setting is also selectable via the GUI. The fifth preset setting allows STR, NTR, SNP linkage analysis on multiple pedigrees to be executed concurrently based on shared alleles.

The present disclosure also relates to the method as described above. Various embodiments and variants disclosed above apply mutatis mutandis to the method.

According to an embodiment, the method is characterized in that the method is used to implement the assay in a plurality of stages, wherein in a first selection stage of the plurality of stages, the method allows selecting a set of features-of-interest from a plurality of features that are configurable using the kit, wherein the plurality of features include exome sequencing preferences and a plurality of custom variants identification modules.

According to an embodiment, the method is characterized in that the method is used to implement the assay in a plurality of stages, wherein in a second wet-lab stage of the plurality of stages, the method allows processing of the genetic material using the kit in accordance with the selected set of features-of-interest in the first selection stage to obtain the genetic DNA readout data from the genetic material, wherein the genetic DNA readout data corresponds to sequencing data, and wherein the kit is used in at least one of a preconception screening, a preimplantation genetic screening, or an application related to assisted reproduction technology, and wherein the genetic material is processed using single cell sequencing.

According to an embodiment, the method is characterized in that the method is used to implement the assay in a plurality of stages, wherein in a third data processing pipeline stage of the plurality of stages, the method allows determination of the occurrence of variants in the DNA readout data in accordance with the selected set of features-of-interest in the first selection stage, wherein the determination of the occurrence of variants in the DNA readout data further comprises:
triggering a specific processing pipeline in accordance with the selected set of features-of-interest in the first selection stage;
executing unique molecular identifier (UMI) demultiplexing on the genetic DNA readout data;
executing mitochondrial (mtDNA) pipeline to measure heteroplasmic variants in the genetic DNA readout data;
detecting short tandem repeats (STR) and VNTR (variable number tandem repeats) in the genetic DNA readout data;
detecting mosaic variants in the genetic DNA readout data;
executing tagging of detected variants that meet gene mode of inheritance (MOI) with expected MOI in a family;
determining whether a detected variant is an inherited variant or a de novo variant; and
auto populating an evidence code when the detected variants match with prestored variant sequences acquired from a specified data source that defines gene variations and corresponding disorders.

According to an embodiment, the method is further characterized in that the method is used to implement the assay in a plurality of stages, wherein in a fourth visualization stage of the plurality of stages, the method allows rendering of a graphical user interface to communicate and interact with results of detection in the third data processing pipeline stage based on a plurality of defined settings.

According to an embodiment, said processing genetic material comprises one, more or all of the following:
(a) extracting said genetic material from a sample taken from a subject;
(b) assessing purity of the extracted genetic material, preferably by measuring UV absorbance thereof;
(c) in case of said genetic material being RNA, reverse transcribing said RNA to obtain cDNA;
(d) in case of said genetic material being DNA or cDNA, shearing or digesting said genetic material to obtain fragments;
(e) enriching protein-coding regions, preferably by hybridizing to complementary oligonucleotides; and
(f) ligating the fragments obtained in (d) to adapters and annealing the ligation products to a solid carrier such as a glass slide.

According to an embodiment, said sample is selected from tissue, biopsy, sample of a fetus, and a bodily fluid, said bodily fluid preferably being blood, throat swab, sputum, surgical drain fluid or amniotic fluid.

According to an embodiment, said genetic material is DNA or RNA, preferably DNA.

In another aspect, an embodiment of the present disclosure provides a system that acquires and processes genomic sequence data to detect copy number variants (CNVs) therein, the system comprising:
an apparatus configured to process at least a portion of a genome of a subject to generate a raw genomic sequence dataset; and
a computing arrangement comprising a data memory device and control circuitry, wherein the control circuitry is configured to:
   acquire the raw genomic sequence dataset from the apparatus and a plurality of candidate CNV detection applications prestored in the data memory device;
   execute a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth;
   combine the baseline CNVs obtained from each of the plurality of candidate CNV detection applications to generate a set of baseline CNVs;
   generate a simulated genomic sequence dataset by simulation of a set of artificial CNVs in at least one target region of the raw genomic sequence dataset by use of a simulation application prestored in the data memory device, wherein the simulated genomic sequence dataset comprises the set of artificial CNVs and the set of baseline CNVs;
   record a location of each artificial CNV of the set of artificial CNVs and each baseline CNV of the set of baseline CNVs in the simulated genomic sequence dataset;
   execute a second CNV calling in the simulated genomic sequence dataset by use of each of the plurality of candidate CNV detection applications;
   eliminate the set of baseline CNVs from CNVs obtained from the second CNV calling in the simulated genomic sequence dataset to obtain a set of new CNVs;
   determine a location of each new CNV of the set of new CNVs in the simulated genomic sequence dataset based on the recorded location of the set of artificial CNVs;
   determine a degree of recall and a degree of precision associated with each of the plurality of candidate CNV detection applications based on a comparison of the location of the set of new CNVs with the location of the set of artificial CNVs;
   select one of the plurality of candidate CNV detection applications as being optimal, based on a combination of the degree of recall and the degree of precision for calling the copy number variants in genomic sequence data; and
   utilize the selected candidate CNV detection application for calling of CNVs in the genomic sequence data.

In another aspect, an embodiment of the present disclosure provides a system that processes a raw genomic sequence dataset to detect one or more copy number variants (CNVs) therein, the system comprising:
a computing arrangement comprising a data memory device and control circuitry, wherein the control circuitry is configured to:
acquire the raw genomic sequence dataset and a plurality of candidate CNV detection applications prestored in the data memory device;
execute a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth;
combine the baseline CNVs obtained from each of the plurality of candidate CNV detection applications to generate a set of baseline CNVs;
generate a simulated genomic sequence dataset by simulation of a set of artificial CNVs in at least one target region of the raw genomic sequence dataset by use of a simulation application prestored in the data memory device, wherein the simulated genomic sequence dataset comprises the set of artificial CNVs and the set of baseline CNVs;
record a location of each artificial CNV of the set of artificial CNVs and each baseline CNV of the set of baseline CNVs in the simulated genomic sequence dataset;
execute a second CNV calling in the simulated genomic sequence dataset by use of each of the plurality of candidate CNV detection applications;
eliminate the set of baseline CNVs from CNVs obtained from the second CNV calling in the simulated genomic sequence dataset to obtain a set of new CNVs;
determine a location of each new CNV of the set of new CNVs in the simulated genomic sequence dataset based on the recorded location of the set of artificial CNVs;
determine a degree of recall and a degree of precision associated with each of the plurality of candidate CNV detection applications based on a comparison of the location of the set of new CNVs with the location of the set of artificial CNVs;
select one of the plurality of candidate CNV detection applications as being optimal, based on a combination of the degree of recall and the degree of precision for calling the copy number variants in genomic sequence data; and
utilize the selected candidate CNV detection application for calling of CNVs in the genomic sequence data.

In another aspect, an embodiment of the present disclosure provides a method for (of) acquiring and processing genomic sequence data to detect copy number variants (CNVs) therein, wherein the method is implemented using a system that comprises an apparatus and a computing arrangement, wherein the method comprises:
processing, by use of the apparatus, at least a portion of a genome of a subject to generate a raw genomic sequence dataset;
acquiring, by use of a control circuitry of the computing arrangement, the raw genomic sequence dataset from the apparatus and a plurality of candidate CNV detection applications prestored in a data memory device of the computing arrangement;
executing, by use of the control circuitry, a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth;
combining, by use of the control circuitry, the baseline CNVs obtained from each of the plurality of candidate CNV detection applications to generate a set of baseline CNVs;
generating, by use of the control circuitry, a simulated genomic sequence dataset by simulation of a set of artificial CNVs in at least one target region of the raw genomic sequence dataset by use of a simulation application prestored in the data memory device, wherein the simulated genomic sequence dataset comprises the set of artificial CNVs and the set of baseline CNVs;
recording, by use of the control circuitry, a location of each artificial CNV of the set of artificial CNVs and each baseline CNV of the set of baseline CNVs in the simulated genomic sequence dataset;
executing, by use of the control circuitry, a second CNV calling in the simulated genomic sequence dataset by use of each of the plurality of candidate CNV detection applications;
eliminating, by use of the control circuitry, the set of baseline CNVs from CNVs obtained from the second CNV calling in the simulated genomic sequence dataset to obtain a set of new CNVs;
determining, by use of the control circuitry, a location of each new CNV of the set of new CNVs in the simulated genomic sequence dataset based on the recorded location of the set of artificial CNVs;
determining, by use of the control circuitry, a degree of recall and a degree of precision associated with each of the plurality of candidate CNV detection applications based on a comparison of the location of the set of new CNVs with the location of the set of artificial CNVs;
selecting, by use of the control circuitry, one of the plurality of candidate CNV detection applications as being optimal, based on a combination of the degree of recall and the degree of precision for calling the copy number variants in genomic sequence data; and
utilizing, by use of the control circuitry, the selected candidate CNV detection application for calling of CNVs in the genomic sequence data.

In yet another aspect, an embodiment of the present disclosure provides a computer program product comprising a non-transitory computer-readable storage medium having computer-readable instructions stored thereon, the computer-readable instructions being executable by a computerized device comprising processing hardware to execute the aforementioned method.

In yet another aspect, an embodiment of the present disclosure provides a method for (of) acquiring and processing genomic sequence dataset to detect one or more copy number variants (CNVs) therein, wherein the method is implemented using a system that comprises a computing arrangement, wherein the method comprises:
acquiring, by use of a control circuitry of the computing arrangement, a raw genomic sequence dataset and a plurality of candidate CNV detection applications prestored in a data memory device of the computing arrangement;
executing, by use of the control circuitry, a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth;
combining, by use of the control circuitry, the baseline CNVs obtained from each of the plurality of candidate CNV detection applications to generate a set of baseline CNVs;
generating, by use of the control circuitry, a simulated genomic sequence dataset by simulation of a set of artificial CNVs in at least one target region of the raw genomic sequence dataset by use of a simulation application prestored in the data memory device, wherein the simulated genomic sequence dataset comprises the set of artificial CNVs and the set of baseline CNVs;
recording, by use of the control circuitry, a location of each artificial CNV of the set of artificial CNVs and each baseline CNV of the set of baseline CNVs in the simulated genomic sequence dataset;
executing, by use of the control circuitry, a second CNV calling in the simulated genomic sequence dataset by use of each of the plurality of candidate CNV detection applications;
eliminating, by use of the control circuitry, the set of baseline CNVs from CNVs obtained from the second CNV calling in the simulated genomic sequence dataset to obtain a set of new CNVs;
determining, by use of the control circuitry, a location of each new CNV of the set of new CNVs in the simulated genomic sequence dataset based on the recorded location of the set of artificial CNVs;
determining, by use of the control circuitry, a degree of recall and a degree of precision associated with each of the plurality of candidate CNV detection applications based on a comparison of the location of the set of new CNVs with the location of the set of artificial CNVs;
selecting, by use of the control circuitry, one of the plurality of candidate CNV detection applications as being optimal, based on a combination of the degree of recall and the degree of precision for calling the copy number variants in genomic sequence data; and
utilizing, by use of the control circuitry, the selected candidate CNV detection application for calling of CNVs in the genomic sequence data.

The present disclosure provides the system and the method that acquires and processes genomic sequence data to detect CNVs. The system comprises the control circuitry that is configured to determine the degree of recall and the degree of precision associated with each of the plurality of candidate CNV detection application that is used for the detection of CNVs in the genomic sequence data. Further, the control circuitry compares the plurality of candidate CNV detection applications based on the degree of recall and the degree of precision associated with each of the plurality of candidate CNV detection application. The control circuitry selects one of the plurality of candidate CNV detection applications as being optimal, based on the combination of the degree of recall and the degree of precision for calling the CNV in genomic sequence data. The selected candidate CNV detection application is utilised for calling of CNVs in the genomic sequence data. Such selected candidate CNV detection application considers the effect of biases introduced in the system due to the use of various capture assay kits and a type of sequencing technique used to generate the genomic sequence data. Notably, the control circuitry is configured to select an optimal CNV detection application for a specific genomic sequence data for detection of CNVs. The selected optimal CNV detection application for the specific genomic sequence data eliminates the effect of biases introduced in the specific genomic sequence data and thereby, enables efficient processing of the specific genomic sequence data to accurately detect new CNVs present therein. The optimal selection of a CNV detection application for each genomic sequence data allows detection of CNVs within each genomic sequence data accurately. Therefore, the system that acquires and processes genomic sequence data is reliable to detect CNVs for any given genomic sequence data. The system is capable of detecting CNVs that cause rare diseases in an individual. For example, some CNVs detected can potentially cause ailments or abnormalities, such as Huntingdon's Disease, which is currently sometimes overlooked due to error in processing and electronic analysis of the genomic sequence data.

The aforementioned system acquires and processes genomic sequence dataset to detect CNVs therein. The system comprises an apparatus configured to process at least a portion of the genome of the subject to generate the raw genomic sequence dataset. The term “copy number variant” or CNV refers to sections of the genome of an individual that are repeated and the number of repeats in the genome varies between individuals in the human population. The “copy number variant” is a result of copy number variation event, which is a type of duplication or deletion event that affects a considerable number of base pairs. Typically, differences in the DNA sequence in genomes contribute to uniqueness of an individual. These differences potentially influence most traits including susceptibility to disease. Since CNVs often encompass genes, the detection of CNVs has important roles both in human disease and drug response. Moreover, in comparison to other genetic variants (e.g. SNPs), CNVs are larger in size and can often involve complex repetitive DNA sequences. In certain cases, CNVs also encompass entire genes, which have a specific protein encoding function ascribed to them. For these reasons, CNVs are potentially more amenable to misinterpretation, and are difficult to detect as compared to other genetic variants.

It will be appreciated that the CNVs are linked with genetic disorders, such as genetic diseases and the like. In human genome, currently most CNVs are found to be benign variants that do not directly cause disease. However, there are several instances where CNVs that affect critical developmental genes and cause rare diseases. For example, there are certain reports of CNVs affecting the nervous system, and contributing to Parkinson's Disease and Alzheimer's Disease. There could be thousands more CNVs in the human population, which lie undetected due to various reasons and problems discussed above. Thus, the system is configured to process the genomic sequence dataset to detect CNVs therein. Subsequently, the accurate and comprehensive detection of CNVs finds applications in decision support and facilitates to pinpoint a target region in the genome that needs to be focused for treatment of the identified rare genetic disorder due to a specific detected CNV, for example, by performing gene therapy. In some cases, certain CNVs could be employed to add discrimination power in forensics.

Throughout the present disclosure, the term “apparatus” refers to a machine or a hardware platform configured to acquire and process a biological sample of the subject (for e.g. a person), specifically, the portion of the genome of the subject. In an example, the apparatus may be a Deoxyribonucleic acid (DNA) readout apparatus, such as a sequencing platform. The sequencing platform may be a large-scale sequencer or a compact benchtop sequencer. Further, throughout the present disclosure, the term “portion of the genome” refers to a stretch of the genome having a given genomic sequence of the subject.

According to an embodiment, the system further comprises a wet-laboratory arrangement, and wherein the wet-laboratory arrangement is configured to process a biological sample of the subject in the wet-laboratory arrangement to derive at least the portion of the genome of the subject to generate the raw genomic sequence dataset. As used herein, the term “wet-laboratory arrangement” refers to a facility, clinic and/or a setup of: instruments, equipment and/or devices used for extraction (invasive or non-invasive), collection, processing, and analysis of body fluid samples; collection, processing, and analysis of genetic material; amplification, enrichment, and processing of genetic material; and analysis of the genetic information received from the amplified genetic material to derive at least the portion of the genome of the subject to generate the raw genomic sequence dataset. Herein the instruments, equipment, and/or devices may include but not limited to centrifuge, ELISA, spectrophotometer, PCR, RT-PCR, High-Throughput-Screening (HTS) system, next generation sequencing systems, Microarray system, Ultrasound, genetic analyzer, deoxyribonucleic acid (DNA) sequencer and SNP analyzer. Notably, in-vitro processing of the biological sample is performed for deriving at least the portion of the genome of the subject to generate the raw genomic sequence dataset. Typically, a standard pipeline process is executed in sequencing to process the biological sample extracted from the subject in the wet-laboratory arrangement in vitro to prepare a sequencing library comprising a plurality of complementary deoxyribonucleic acid (cDNA) fragment molecules. Moreover, the biological sample of the subject refers to a laboratory specimen taken, preferably non-invasively by sampling under controlled environments, that is, gathered matter of a medical subject's tissue, fluid, or other material derived from the subject. Examples of the biological sample include, but are not limited to, blood, throat swabs, sputum, surgical drain fluids, tissue biopsies, amniotic fluid, or sample of fetus.

According to an embodiment, the wet-laboratory arrangement processes the biological sample of the subject to isolate DNA (or RNA), determine a presence of cell-free DNA (cfDNA) fragments therein, in order to prepare the sequencing library and further to sequence the isolated genetic material. The term “cell-free DNA” refers to DNA that is not within a cell. Herein, the wet-laboratory arrangement extracts the cell-free DNA (cfDNA) present in the biological sample and obtains DNA fragments. In an example, in order to execute next generation sequencing (NGS), an input sample, such as a sample of DNA of a subject that is isolated from the subject. For example, after sampling blood, a small amount of DNA is isolated from the sampled blood. The quantity of isolated DNA is insufficient for sequencing library preparation. Therefore, the input sample is then fragmented into short sections. The length of these sections is optionally same, for example, less than 250 base pairs, optionally in a range of 100 to 250 base pairs. The length optionally also depends on a type of sequencing machine used or a type of experiment to be conducted. In some cases where the length of DNA sections is relatively longer, for example longer than 250 base pairs, the fragments are ligated with generic adaptors (i.e. small piece of known DNA located at the read extremities) and annealed to a glass slide using the adaptors (e.g. in Illumina based sequencing). In some cases, mRNA transcripts are isolated which correspond to the coding regions of functional genes, for example in exome sequencing.

According to an embodiment, the apparatus is further configured to execute sequencing of the plurality of complementary deoxyribonucleic acid (cDNA) fragment molecules concurrently in a next generation sequencing (NGS) process to generate the raw genomic sequence dataset. Notably, sequencing, for example, DNA sequencing, is the process of determining the sequence of nucleotides in a given section of DNA. An example of the NGS process is described below.

In an example, in NGS, vast numbers of short reads (e.g. the plurality of cDNA fragment molecules) are sequenced in a single run. After the sequencing library is prepared, PCR is carried out to amplify each read, creating a spot with many copies of the same read. The amplified copies are then separated into single strands by denaturation for subsequent sequencing. In NSG, the sequencing is done in parallel manner using sequencing-by-synthesis, to produce a set of concurrent data, composed of millions of short sequencing reads. Thus, the slide is covered with large quantity of nucleotides and DNA polymerase. Such nucleotides are fluorescently labelled, with unique colour for a base (for example, different colour for different nucleic acid bases, i.e. A, T, C, and C). The fluorescently labelled base has a terminator, so that only one base is added at a time. Since one base is added at a time, this enables to capture an image of the slide. A fluorescent signal in each read location indicates a particular base that is recently added. The slide is then prepared for a next cycle. The terminators are automatically removed, allowing the next base to be added, and the fluorescent signal is removed, preventing the signal from contaminating the next image. The process is repeated, adding one nucleotide at a time and imaging in between. A computing device, such as the computing arrangement, is then employed to detect a base at each read location site in each image, which is then used to construct a sequence. The readout of the sequence by the apparatus corresponds to the raw genomic sequence dataset (or readout). Typically, the raw genomic sequence dataset derived from the biological sample includes biases (or stochastic data errors). Beneficially, the system described herein provides significantly accurate results despite the biases in the raw genomic sequence dataset. As an alternative to the NGS, long-read sequencing may also be applicable.

According to an embodiment, the apparatus is configured to perform at least one of an exome sequencing or whole genome sequencing (WGS), to generate the raw genomic sequence dataset. The apparatus is a sequencing platform that is used to perform the exome sequencing, to generate the raw genomic sequence dataset. The term ‘exome’ refers to a complete sequence of all exons in protein-coding genes in the genome. Alternatively, depending on user-preference, WGS may be executed to generate the raw genomic sequence dataset. In an example, the WGS utilizes a large whole genome (e.g. a human genome) for generating the raw sequencing dataset. Optionally, the apparatus is potentially used to perform a small whole-genome sequencing (e.g. microbe), a targeted gene sequencing (amplicon, gene panel), a whole-transcriptome sequencing, a gene expression profiling with mRNA-sequencing, or a targeted gene expression profiling.

Furthermore, the system comprises the computing arrangement comprising the data memory device and the control circuitry. Notably, the term “computing arrangement” refers to a structure and/or hardware module that includes programmable and/or non-programmable components that are configured to store, process and/or share the biological information, such as the raw sequence dataset related to the genome of the subject. Moreover, it will be appreciated that the computing arrangement is optionally implemented as a single hardware computing device, such as a server, or plurality of hardware computing devices operating in a parallel or distributed architecture. In an example, the computing arrangement optionally includes components such as the data memory device, a processor, a display, a network interface and the like, to store, process and/or share information with other computing components, such as a user device/user equipment. Examples of the computing arrangement include, but are not limited to, a medical system, a server, an electronic device, a piece of specialized computational biology equipment, or other computing devices. Optionally, the computing arrangement is part of a machine (i.e. integrated into the apparatus). The term “data memory device” as used herein refers to a non-transitory computer-readable storage medium that stores data. In an example, the data memory device is a volatile data memory. In another example, the data memory device is a combination of rapid-access memory (for example, solid-state data memory) and persistent memory (for example, optical disc drive, magnetic hard disc data memory) to store data currently being used by the computing arrangement. Examples of the data memory device include, but is not limited to random access memory (RAM), synchronous dynamic random-access memory (SDRAM), dynamic RAM (DRAM), Dual In-line Memory Module (DIMM), video random access memory (VRAM), graphic double-data-rate (GDDR) RAM, ROM, and the like.

Moreover, the term “control circuitry” refers to a computational element that is operable to respond to and processes instructions that drive the aforementioned system. Optionally, the control circuitry includes, but is not limited to, a microprocessor, a microcontroller, a complex instruction set computing (CISC) microprocessor, an application-specific integrated circuit (ASIC), a reduced instruction set (RISC) microprocessor, a very long instruction word (VLIW) microprocessor, or any other type of processing or control circuitry. Furthermore, the control circuitry may refer to one or more individual processors, processing devices, a processing unit that is part of a machine, and various elements associated with the system. Optionally, the control circuitry and the data memory device are communicatively coupled to each other.

Moreover, the control circuitry is configured to acquire the raw genomic sequence dataset from the apparatus and a plurality of candidate CNV detection applications prestored in the data memory device. The control circuitry is communicatively coupled to the apparatus to acquire the raw genomic sequence dataset generated by the apparatus. The term “plurality of candidate CNV detection applications” refers to different applications that potentially detect CNVs but vary in their performance in terms of precision and recall. In an example, the different applications are different software applications, algorithms, or a plurality of executable codes. Examples of the plurality of candidate CNV detection applications include, but are not limited to regression-based CNV detection application, read depth data-based CNV detection application, and the like. Some examples of CNV detection applications include “CANOES”, “Dragen™”, “ExomeDepth”, “Sentieon” and so forth. The CANOES is a CNV detection application that detects the CNVs by using a negative binomial distribution and estimation of variance of read sequences using a regression-based approach based on selected reference samples in a given genomic sequence dataset. The Dragen™ is a CNV detection application that maps, aligns, sorts and duplicates CNVs. The ExomeDepth is a CNV detection application that uses read depth data to call CNVs from exome sequencing experiments.

The different CNV detection applications are stored as candidate applications (i.e. a plurality of candidate CNV detection applications) in the data memory device that are retrieved by the control circuitry to process the raw genomic sequence dataset acquired from the apparatus. In an example, the control circuitry is configured to retrieve the plurality of candidate CNV detection applications that are stored in the data memory device one at a time. In another example, the control circuitry is configured to retrieve all the candidate CNV detection application of the plurality of candidate CNV detection applications at once (i.e. concurrent/parallel processing), and then process the raw genomic sequence dataset using each of the retrieved plurality of candidate CNV detection application.

Furthermore, the control circuitry is configured to execute a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth. The term “CNV calling” used herein refers to a process to identify copy number variants from the raw genomic sequence dataset. Optionally, the CNV calling is carried out in a plurality of steps. In a first step, exome sequencing or WGS is carried out by the apparatus to create files in a FASTQ format. The FASTQ (also referred to as Fastq) is a common format that is employed for storing next generation sequencing (NGS) data. In a second step, the obtained sequences in the first step are aligned to a reference genome to create files in a Binary Alignment Map (BAM) file format. In a third step, identification of a difference of the aligned reads from the reference genome is carried out. The third step facilitates in further processing for identification of the copy number variants in the raw genomic sequence dataset. The first CNV calling is utilized in downstream processing of the raw genomic sequence dataset for the purpose of comprehensive detection of CNVs. The baseline CNVs refer to naturally occurring CNVs that are known to be present in the raw genomic sequence dataset and are called from the plurality of candidate CNV detection applications. Since the baseline CNVs are known to be existent, the baseline CNVs are recognized as ground truth for comparison of the performance of the plurality of candidate CNV detection applications. The control circuitry utilizes each candidate CNV detection application of the plurality of candidate CNV detection applications to execute the first CNV calling in the randomly selected regions of the raw genomic sequence dataset to obtain baseline CNVs from each of the plurality of candidate CNV detection application. Notably, the obtained baseline CNVs from each of the plurality of candidate CNV detection application may or may not be the same.

Moreover, the control circuitry is configured to combine the baseline CNVs obtained from each of the plurality of candidate CNV detection applications to generate a set of baseline CNVs. The baseline CNVs obtained from each of the plurality of candidate CNV detection applications may be different in number and/or their respective locations in the randomly selected regions of the raw genomic sequence dataset. The control circuitry combines the results obtained from each candidate CNV detection application to form set of baseline CNVs (i.e. collection of baseline CNVs obtained from all of the plurality of candidate CNV detection applications), such that each obtained baseline CNV occurs only once in the set of baseline CNVs. For example, the baseline CNVs obtained from a first candidate CNV detection application are CNV1, CNV2 and CNV3. The baseline CNVs obtained from a second candidate CNV detection application are CNV1, CNV2, CNV3 and CNV4. The baseline CNVs obtained from a third candidate CNV detection application are CNV1 and CNV3. The control circuitry combines the obtained baseline CNVs CNV1, CNV2, CNV3 and CNV4 to obtain the set of baseline CNVs recognized as the ground truth.

Furthermore, the control circuitry is configured to generate a simulated genomic sequence dataset by simulation of a set of artificial CNVs in at least one target region of the raw genomic sequence dataset by use of a simulation application prestored in the data memory device. The simulated genomic sequence dataset comprises the set of artificial CNVs and the set of baseline CNVs. The “target region” of the raw genomic sequence dataset refers to one or more areas of interest (e.g. focus gene panels) for sequencing in the raw genomic sequence dataset. With reference to the present disclosure, the target region may be areas in which the presence of abnormalities due to the CNVs may lead to pathogenesis. For example, the target region may be an area corresponding to exons in the raw genomic sequence dataset, i.e. the certain coding regions of interest in the genome. The information about the presence of one or more CNVs in the target regions of the genome of the subject is potentially used for decision support so as to assist in the identification of the occurrence of rare genetic disorders in the subject due to the identified one or more CNVs. Thus, the control circuitry simulates the set of artificial CNVs in at least one target region of the raw genomic sequence dataset for identification of the CNVs that may be responsible for the occurrence of rare genetic disorders. The term “simulation application” refers to a framework that is configured to run and simulate the set of artificial CNVs for evaluation of the plurality of candidate CNV detection application. The control circuitry utilizes the simulation application prestored in the data memory device for the simulation of the set of artificial CNVs, such that the artificial CNVs are generated in the target region of the raw genomic sequence dataset. Since the set of artificial CNVs is simulated in the raw genomic sequence dataset that comprises the called set of baseline CNVS, therefore, the simulated genomic sequence dataset comprises the set of artificial CNVs simulated by the simulation application and the set of baseline CNVs called during the first CNV calling by the control circuitry. Notably, the target region of the raw genomic sequence dataset may overlap with the randomly selected regions of the raw genomic sequence dataset.

Optionally, the simulation application is a “Ximmer” tool. The “Ximmer” tool is an analysis pipeline that automatically configures and runs a variety of CNV detection applications. The “Ximmer” tool acts as a simulation application that can create artificial CNVs in sequencing data. The “Ximmer” tool is potentially utilized as a visualization and curation tool that can combine results from multiple CNV detection applications and allow a user to inspect them, along with relevant annotations.

Moreover, the control circuitry is configured to record a location of each artificial CNV of the set of artificial CNVs and each baseline CNV of the set of baseline CNVs in the simulated genomic sequence dataset. The locations of the each artificial CNV and each baseline CNV in the simulated genomic sequence dataset is recorded by the control circuitry that is used as a reference for measurement of performance of the plurality of candidate CNV detection applications at a later stage. The locations of each of the baseline CNV of the set of baseline CNVs are known, and therefore the location of each baseline CNV may be reliably used as a reference. Further, the artificial CNVs are simulated at predefined target regions, whose locations are known to the simulation application. The locations of each artificial CNV of the set of artificial CNVs and each baseline CNV of the set of baseline CNVs in the simulated genomic sequence dataset are stored in a database. Notably, the database is a part of the data memory device.

Furthermore, the control circuitry is configured to execute a second CNV calling in the simulated genomic sequence dataset by use of each of the plurality of candidate CNV detection applications. The control circuitry utilizes each candidate CNV detection application of the plurality of candidate CNV detection applications to execute the second CNV calling in the simulated genomic sequence dataset to obtain CNVs, such as the set of baseline CNVs and the set of artificial CNVs present in the simulated genomic sequence dataset. Notably, the set of baseline CNVs and the set of artificial CNVs obtained from each of the plurality of candidate CNV detection application may or may not be the same. It will be appreciated that the CNVs called during the execution of the second CNV calling may comprise one or more baseline CNVs which are potentially undetected during the execution of the first CNV calling. It will be further appreciated that the CNVs called during the execution of the second CNV calling may comprise one or more CNVs other than the simulated artificial CNVs present in the set of the artificial CNVs.

Moreover, the control circuitry is configured to eliminate the set of baseline CNVs from CNVs obtained from the second CNV calling in the simulated genomic sequence dataset to obtain a set of new CNVs. The set of new CNVs obtained from the second CNV calling in the simulated genomic sequence dataset may comprise the set of artificial CNVs and the one or more CNVs other than the simulated artificial CNVs after elimination of the set of baseline CNVs.

Furthermore, the control circuitry is configured to determine a location of each new CNV of the set of new CNVs in the simulated genomic sequence dataset based on the recorded location of the set of artificial CNVs. A sequence of a new CNV of the set of new CNVs in the simulated genomic sequence dataset is compared with sequences of each artificial CNV of the set of artificial CNVs to determine a location of the new CNV of the set of new CNVs in the simulated genomic sequence dataset. Similarly, the comparison of the sequences of each new CNV of the set of new CNVs is performed with the sequences of each artificial CNV of known locations to determine the locations of the set of new CNVs.

Moreover, the control circuitry is configured to determine a degree of recall and a degree of precision associated with each of the plurality of candidate CNV detection applications based on a comparison of the location of the set of new CNVs with the location of the set of artificial CNVs. The control circuitry compares the performance of each of the plurality of candidate CNV detection application in determining accurate locations of the set of new CNVs in the simulated genomic sequence dataset. Further, based on the performance, the control circuitry determines the degree of recall and the degree of precision associated with each of the plurality of candidate CNV detection application.

According to an embodiment, the control circuitry is further configured to determine the degree of recall associated with each of the plurality of candidate CNV detection applications by identification of a true positive, if a location of a new CNV of the set of new CNVs and a corresponding location of an artificial CNV of the set of artificial CNVs matches. A new CNV detected is considered as true positive if the location of the new CNV is the same (or almost same) to the corresponding location of the artificial CNV in the simulated genomic sequence dataset. In an example, a candidate CNV detection application performs a second CNV calling to obtain the new CNVs. In such a case, let a sequence of an artificial CNV may be ‘ATTCGAC’ at a location L1 in the simulated genomic sequence dataset. The control circuitry identifies a true positive, if the location of a sequence ‘ATTCGAC’ of a new CNV matches with the location L1 of the sequence ‘ATTCGAC’ of an artificial CNV.

The control circuitry is further configured to determine the degree of recall associated with each of the plurality of candidate CNV detection applications by identification of a false positive, if a location of a new CNV of the set of new CNVs is detected at a location that is different than a location of an artificial CNV of the set of artificial CNVs. A new CNV detected is considered as the false positive if the location of a new CNV of the set of new CNVs is detected at a location that is different than a location of an artificial CNV of the set of artificial CNVs. In an example, a candidate CNV detection application performs a second CNV calling to obtain the new CNVs. In such a case, let a sequence of an artificial CNV may be ‘TCCGAACTG’ at a location L1 in the simulated genomic sequence dataset. The control circuitry identifies a false positive, if a location of a new CNV having a sequence ‘TCCGAACTG’ is detected at a location (e.g. a location L2) that is different than a location L1 of the sequence ‘TCCGAACTG’ of an artificial CNV of the set of artificial CNVs.

The control circuitry is further configured to determine the degree of recall associated with each of the plurality of candidate CNV detection applications by identification of a false negative, if no new CNV of the set of new CNVs is detected at a location of an artificial CNV of the set of artificial CNVs. In other words, a new CNV detected is considered as a false negative, if no new CNV of the set of new CNVs is detected at a location of an artificial CNV of the set of artificial CNVs. It will be appreciated that a total number of CNVs detected by a candidate CNV detection application in the simulated genomic sequence dataset is equal to the true positives and the false negatives associated with the candidate CNV detection application. The control circuitry is further configured to determine a higher degree of recall associated with a candidate CNV detection application having a greater number of true positives than a candidate CNV detection application having a lesser number of true positives. In an example, three candidate CNV detection applications A, B and C are used to call the CNVs in a genomic sequence dataset. The candidate CNV detection application A identifies 5 CNVs in the genomic sequence dataset, thus, it is assigned 5 true positives. The candidate CNV detection application B identifies 8 CNVs in the genomic sequence dataset, thus, it is assigned 8 true positives. The candidate CNV detection application C identifies 3 CNVs in the genomic sequence dataset, thus, it is assigned 3 true positives. Therefore, the control circuitry determines the degree of recall associated with the candidate CNV detection application B the highest and the control circuitry determines the degree of recall associated with the candidate CNV detection application C the lowest amongst the three candidate CNV detection applications.

According to an embodiment, the control circuitry is further configured to measure an extent of overlap of a location of a new CNV of the set of new CNVs with a corresponding location of an artificial CNV of the set of artificial CNVs, for determination of the degree of precision associated with each of the plurality of candidate CNV detection applications. In other words, the degree of precision associated with a plurality of candidate CNV detection application is a measure of the exactness of a determined location of the new CNV with respect to the corresponding location of an artificial CNV. For example, a sequence of a detected new CNV of the set of new CNVs may be ‘AGGTCCAGC’. If a candidate CNV detection application detects the location of the new CNV having the sequence ‘AGGTCCAGC’ to be precisely overlapping with a location of an artificial CNV having a sequence ‘AGGTCCAGC’, then the control circuitry determines the degree of precision associated with the plurality of candidate CNV detection application as high.

According to an embodiment, the control circuitry is further configured to set a specified threshold for determination of the extent of overlap of the location of the new CNV of the set of new CNVs with the corresponding location of the artificial CNV of the set of artificial CNVs. The specific threshold is a measure of a minimum extent of overlap of the location of the new CNV of the set of new CNVs with the corresponding location of the artificial CNV of the set of artificial CNVs, such that if the extent of overlap of the location of the new CNV is more than the specified threshold, then the location of the new CNV is said to be matched with the corresponding location of the artificial CNV. Optionally, the specified threshold of 50% is set for determination of the extent of overlap of the location of the new CNV of the set of new CNVs with the corresponding location of the artificial CNV of the set of artificial CNVs. In such a case, if a candidate CNV detection application detects an extent of overlap of a location of the new CNV to be 50% (i.e. a 50% match or overlap) or more with a corresponding location of the artificial CNV, the location of the new CNV is said to be matched with the corresponding location of the artificial CNV.

According to an embodiment, the control circuitry is configured to allocate a highest degree of precision to a first candidate CNV detection application among the plurality of candidate CNV detection applications, based on the measured extent of overlap of the location of the new CNV of the set of new CNVs with the corresponding location of the artificial CNV of the set of artificial CNVs, by use of each of the plurality of candidate CNV detection applications. Notably, larger the extent of overlap detected by the candidate CNV detection application, higher is the degree of precision associated with therewith. In an example, an extent of overlap measured by a first candidate CNV detection application is 80%, an extent of overlap measured by a second candidate CNV detection application is 67% and an extent of overlap measured by a third candidate CNV detection application is 70%. Thus, the degree of precision associated with the first candidate CNV detection application is the highest and the degree of precision associated with the second candidate CNV detection application is the lowest.

Furthermore, the control circuitry is configured to select one of the plurality of candidate CNV detection applications as being optimal, based on a combination of the degree of recall and the degree of precision for calling the copy number variants in genomic sequence data. A candidate CNV detection application of the plurality of candidate CNV detection applications is selected as optimal that has the highest degree of recall and the highest degree of precision associated therewith. However, optimal candidate CNV detection application may also be selected based on a compromise between the degree of recall and the degree of precision depending upon its usage in various applications. The optimal candidate CNV detection application for a specific genomic sequence data is selected to be used for calling the copy number variants in that genomic sequence data in order to provide optimal results, i.e. facilitating an optimal calling of the copy number variants in the genomic sequence data.

According to an embodiment, the control circuitry is further configured to generate a precision-recall curve relationship associated with each of the plurality of candidate CNV detection applications, and wherein the selection of one of the plurality of candidate CNV detection applications as optimal depends upon a balance between the degree of recall and the degree of precision. The balance between the degree of recall and the degree of precision related to each of the plurality of candidate CNV detection applications is indicated by a corresponding area-under-precision-recall-curve in the generated precision-recall curve relationship. The detection of the new CNVs may be scored and used to create precision-recall curve relationship. Optionally, the precision-recall curve relationship is displayed as a graphical precision-recall curve plot. The precision-recall curve relationship is a measure of performance of each of the candidate CNV detection application. The precision-recall curve relationship depicts a change in degree of recall and the degree of precision associated with a candidate CNV detection application with a change in a measure of sensitivity associated therewith. Using such precision-recall curve relationships, precision-recall-curve conveniently and accurately identifies the optimal candidate CNV detection application. The optimal candidate CNV detection application is selected by choosing the precision-recall-curve having a maximum area-under-precision-recall-curve. Alternatively, some applications that require CNV detection potentially prioritize the degree of precision over the degree of recall, or vice versa. Thus, the selection process of the optimal candidate CNV detection application is executed by differential weighting of the degree of precision and the degree of recall based upon the application for which the candidate CNV detection application is used.

Moreover, the control circuitry is configured to utilize the selected candidate CNV detection application for calling of CNVs in the genomic sequence data. The control circuitry is configured to utilize the optimal candidate CNV detection application for accurate calling of the CNVs in the genomic sequence data. The accurate detection of CNVs by the control circuitry of the system provides decision support to enable recognition of ailments or abnormalities in the genomic sequence data of an individual. Moreover, the recognition of ailments or abnormalities facilitates a subsequent treatment of the identified ailments or abnormalities, for example, by performing gene therapy.

The present disclosure also relates to the method as described above. Various embodiments and variants disclosed above apply mutatis mutandis to the method.

According to an embodiment, the method further comprising determining, by the control circuitry, the degree of recall associated with each of the plurality of candidate CNV detection applications by identifying:
a true positive, if a location of a new CNV of the set of new CNVs matches with a corresponding location of an artificial CNV of the set of artificial CNVs;
a false positive, if a location of a new CNV of the set of new CNVs is detected at a location that is different than a location of an artificial CNV of the set of artificial CNVs; and
a false negative, if no new CNV of the set of new CNVs is detected at a location an artificial CNV of the set of artificial CNVs.

According to an embodiment, the method further comprises measuring, by use of the control circuitry, an extent of overlap of a location of a new CNV of the set of new CNVs with a corresponding location of an artificial CNV of the set of artificial CNVs, for determination of the degree of precision associated with each of the plurality of candidate CNV detection applications.

According to an embodiment, the method further comprises allocating, by use of the control circuitry, a highest degree of precision to a first candidate CNV detection application among the plurality of candidate CNV detection applications, based on the measured extent of overlap of the location of the new CNV of the set of new CNVs with the corresponding location of the artificial CNV of the set of artificial CNVs, by use of each of the plurality of candidate CNV detection applications.

According to an embodiment, the method further comprises setting, by use of the control circuitry, a specified threshold for determination of the extent of overlap of the location of the new CNV of the set of new CNVs with the corresponding location of the artificial CNV of the set of artificial CNVs.

According to an embodiment, the method comprises generating, by use of the control circuitry, a precision-recall curve relationship associated with each of the plurality of candidate CNV detection applications, and wherein the selection of one of the plurality of candidate CNV detection applications as optimal depends upon a balance between the degree of recall and the degree of precision, wherein the balance between the degree of recall and the degree of precision related to each of the plurality of candidate CNV detection applications is indicated by a corresponding area-under-precision-recall-curve in the generated precision-recall curve relationship.

### DETAILED DESCRIPTION OF THE DRAWINGS

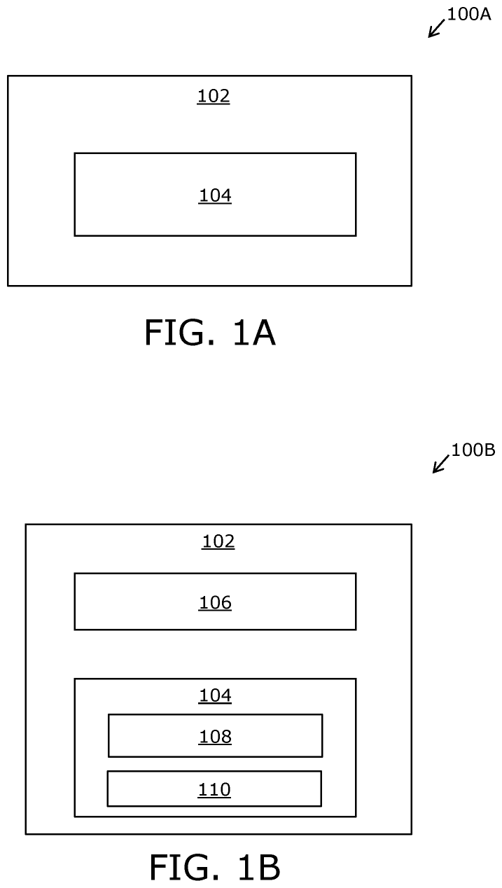

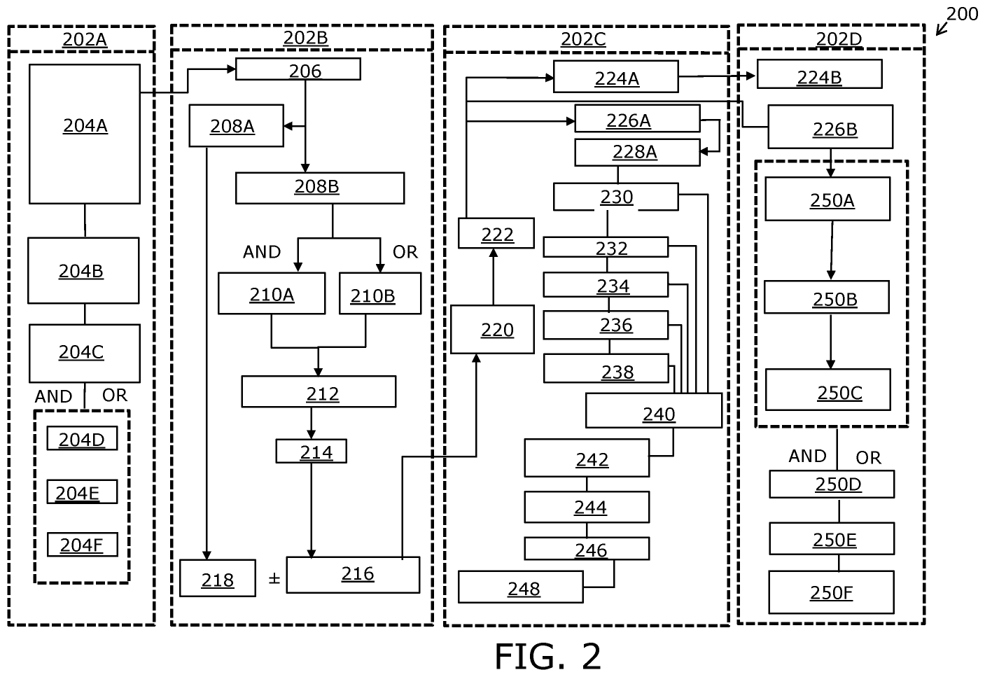

Referring to FIG. 1A, there is shown a block diagram 100A of a kit 104 used in an apparatus 102, in accordance with an embodiment of the present disclosure. The kit 104, when in operation, performs a wet-lab assay. The assay includes processing genetic material that is derived from one or more cell exomes. The assay detects single nucleotide variants (SNVs), indels and copy number variants (CNVs) in genetic DNA readout from the genetic material. The kit 104 is executable as a single assay that processes the genetic material to obtain genetic DNA readout. The kit 104 includes a software product (not shown) that is executable on a computing hardware (not shown) to cause the computing hardware to invoke algorithms to process the genetic DNA readout by comparing portions of the genetic DNA readout against one or more DNA sequence transcripts, to determine an occurrence of variants corresponding to the one or more DNA sequence transcripts in the DNA readout data.

The algorithms invoked by the computing hardware include an algorithm for detecting both SNVs and CNVs, and optionally indels, in the genetic DNA readout from the genetic material. The computing hardware further invokes an algorithm for annotating clinically relevant CNVs present in the genetic DNA readout from the genetic material. The computing hardware further invokes an algorithm that prioritizes one or more portions of the genetic DNA readout from the genetic material depending on phenotype associated with the one or more portions. Furthermore, the computing hardware further invokes an algorithm that detects variant calling for pharmacogenomic (PGx) markers and sample tracking SNPs.

Referring to FIG. 1B, there is shown a block diagram 100B of a kit 104 used in an apparatus 102, in accordance with another embodiment of the present disclosure. In this embodiment, the apparatus further includes a computing hardware 106. The Kit 104 further includes a software product 108 and a genetic material processing arrangement 110.

The kit 104, when in operation, performs a wet-lab assay. The assay includes processing genetic material that is derived from a cell exome (e.g. by single cell sequencing). The kit 104 finds application in a preconception screening, a preimplantation genetic screening, or an application related to assisted reproduction technology. In this embodiment, the genetic material processing arrangement 110 is used to process the genetic material to obtain genetic DNA readout. The assay detects single nucleotide variants (SNVs), indels and copy number variations (CNVs) in genetic DNA readout from the genetic material. The kit 104 is executable as a single assay that processes the genetic material to obtain genetic DNA readout. The software product 108 of the kit 104 is executable on the computing hardware 106 to cause the computing hardware 106 to process the genetic DNA readout by comparing portions of the genetic DNA readout against DNA sequence transcripts, to determine an occurrence of variants corresponding to the DNA sequence transcripts in the DNA readout data.

The software product 108 of the kit 104 is executable on the computing hardware 106 to cause the computing hardware 106 to detect both SNVs and CNVs in the genetic DNA readout from the genetic material; annotate clinically relevant CNVs present in the genetic DNA readout from the genetic material; prioritize one or more portions of the genetic DNA readout from the genetic material depending on phenotype associated with the one or more portions; and detect variant calling for pharmacogenomic (PGx) markers and sample tracking SNPs.

It will be appreciated by a person skilled in the art that the FIGS. 1A and 1B include a simplified illustration of the system 100A and 100B for the sake of clarity only, which should not unduly limit the scope of the claims herein. The person skilled in the art will recognize many variations, alternatives, and modifications of embodiments of the present disclosure.

Referring to FIG. 2, there is shown an exemplary scenario 200 for implementation of an exemplary kit to perform a bespoke wet-lab assay, in accordance with an embodiment of the present disclosure. The exemplary scenario 200 includes four sequential stages, namely a first selection stage 202A, a second wet-lab stage 202B, a third data processing stage 202C, and a fourth visualization stage 202D.

The first selection stage 202A refers to a selection stage in which an entity that uses a kit is able to select a set of features-of-interest as per customized requirements (i.e. a bespoke clinical exome assay configurable as per requirement for a particular vendor, entity, or an end-user). The second wet-lab stage 202B refers to genetic material processing stage using the kit in accordance with the selected set of features-of-interest in the first selection stage 202A to obtain genetic DNA readout from the genetic material. The third data processing stage 202C refers to data processing pipelines in which the output (i.e. the genetic DNA readout) from the second wet-lab stage 202B is processed in accordance with the selected set of features-of-interest in the first selection stage 202A. The fourth visualization stage 202D refers to the visualization stage in which a graphical user interface is rendered for visualization and further analysis of the processed data at the third data processing stage 202C.

In the first selection stage 202A, when a user (on the purchase or optionally after the purchase of the kit), has options to choose features-of-interest as per requirement. The kit allows data processing, variant filtering, variant prioritization, and visualization of processed data. In this exemplary scenario 200, at a step 204A, the data processing features and visualization features are configurable and are made available to the owner of the kit as per requirement. In this embodiment, a token provides access to or activates certain selected features (or modules). At a step 204B, exome sequencing preferences are selected, i.e. a whole exome sequencing (WES), a shallow whole-genome sequencing (sWGS), or combination thereof (i.e. WES±sWGS or sWGS±WES). At a step 204C, an exome plus analysis feature is selected. In addition to the exome sequencing preferences, following features are selectable (i.e. allowed to opt-in or opt-out) as per choice: i) a prenatal module 204D; ii) early-infantile epileptic encephalopathy (EIEE) neuro-medical module 204E; and a carrier screening panel module 204F.

In the second wet-lab stage 202B, at a step 206, a DNA sample is extracted locally. At a step 208A, a sample tracking assay of choice (i.e. as per selection performed in the first selection stage 202A) is run locally. At a step 208B, the DNA sample is sheared (enzymatic shearing or an acoustic shearing). At a step 210A, the fragmented DNA samples after shearing are used to prepare a sWGS (shallow-low-level) library that incorporates unique molecular identifiers (UMI) and an index of a corresponding sample (i.e. a sample index) in case the sWGS feature is selected in the sequence preferences in the first selection stage 202A. At a step 210B, the fragmented DNA samples after shearing are used in a WES library preparation that also incorporate UMI and sample index in case the WES feature is selected in the sequence preferences in the first selection stage 202A. At a step 212, the sWGS and the WES libraries are pooled (i.e. combining sWGS and the WES libraries) for high-coverage paired-end exome sequencing (which enables full exome plus downstream analysis).

At a step 214, sequencing of pooled libraries is performed. In this case, the sequencing is performed using a defined number of base pairs (bp) paired end reads (short reads via next generation sequencing (NGS)). Long-read sequencing may be applied as an alternative. At a step 216, the sequencing data obtained from the sequencing is uploaded to a cloud-based sequence analysis and visualization platform communicatively coupled to the kit. In this embodiment, the sequencing data uploaded is in the form of BCL, FASTQ, BAM, VCF or BED format. The sequencing data is uploaded along with interpretation request (IR) that indicate selected token(s) that provide access to selected modules (i.e. features) in the first selection stage 202A. At a step 218, the output of the sample tracking assay that includes SNP data for tracking performed at the step 208A, is also uploaded in the cloud-based sequence analysis and visualization platform.

In the third data processing stage 202C, the data processing pipeline stage begins in which the uploaded sequencing data is processed. At a step 220, a specific processing pipeline(s) is triggered in accordance w the selected features (i.e. selected module in form of token) in the first selection stage 202A. At a step 222, an initial alignment of the sequencing data is performed with reference genomic dataset. The sequencing data is aligned to a latest version of genome build assembly (in this case, the GRCh38/hg38 human genome build assembly is used). This alignment enables to identify meaningful variation in an individual's genome sequence to distinguish what is healthy from what is potentially pathological. At a step 224A, using the alignment data at a step 222 or raw sequencing data uploaded, sample tracking SNPs with quality control are generated. The SNPS and in some cases short tandem repeat markers are used for genetic sample tracking to avoid sample mix-ups. At a step 226A, UMI demultiplexing is performed on the sequencing data (i.e. on the raw sequencing data uploaded or the alignment data obtained at step 222). At step 228A, using the alignment data at the step 222 or raw sequencing data, mitochondrial (mtDNA) pipeline is executed to measure heteroplasmy (i.e. heteroplasmic variants) and to recognize the most functionally important mitochondrial variants that contribute to phenotype (e.g. a disease) among a huge number of candidates. The mtDNA data is extracted from the sequencing data (i.e. from sWGS and WES data). In an implementation, the steps 224A, 226A, and 228A are performed concurrently. In another implementation, the steps 224A, 226A, and 228A are performed one after another in any defined order.

At a step 2248, in the fourth visualization stage 202D, the sample tracking SNPs with quality control generated at the step 222A, are rendered on a GUI (i.e. a visual interface). The GUI is rendered on an apparatus (not shown). At a step 2268, the GUI allows setting configurations to control the data processing operations at the third data processing stage 202C. The results of various data processing operations executed at the third data processing stage 202C are rendered on the GUI for further analysis, and also the data processing is performed based on the plurality of defined settings (i.e. preset settings), specified knowledgebase, and panels selected and applied via the rendered GUI. The third data processing stage 202C and the fourth visualization stage 202D are executed in synchronization to each other. In this exemplary scenario 200, a first preset setting 250A of the plurality of preset settings (preset 1) when selected allows to preload primary gene panel(s) and associated data (e.g. the prenatal module 204D or EIEE module panel 204E). In a case where no identifiable pathogenic variant is detected by the primary panels based on predefined rules, a second preset setting 250B (preset 2) of the plurality of preset settings is applied. In the second preset setting 250B, Mendelian inheritance (e.g. OMIM or MORBID) data, and HPO data are preloaded and rendered alongside the preload primary gene panel(s) and associated data.

Now referring back to third data processing stage 202C, at a step 230, the duplication and deletion variants in the DNA readout data are detected. At a step 232, a copy number variation (CNV) calling is executed. Alternatively, both SNVs and CNVs are detected together in the genetic DNA readout using an algorithm. Additionally, variant calling for pharmacogenomic (PGx) markers is also executed. At a step 234, a SNV and indel calling is executed. At a step 236, a STR and VNTR calling is executed. At a step 238, mosaic variants are detected. At a step 240, the different variants called (duplication and deletion variants including further CNV calling, SNV, indel, STR, and VNTR, are tagged as per the type of variant at the corresponding site on the genetic DNA readout data and visualized via the GUI. The tagging (or annotations) is performed for the variants that meet gene mode of inheritance (MOI) (i.e. observed gene MOI) with expected MOI in a family. At a step 242, it is determined whether a variant is an inherited variant or a de novo variant. At a step 244, the detected variants are categorized on primary gene panel(s) (i.e. variant tiering is performed). At a step 246, variant prioritization is performed for all the detected variants based on genes-of-interest. At a step 248, ACMG evidence code is auto populated in case the detected variants match with the ACMG provided variant sequences. The ACMG stands for the American College of Medical Genetics and Genomics that has published recommendations for reporting incidental findings in the exons of certain genes (typically 59 genes are prescribed).

In the fourth visualization stage 202D, as discussed above, the results of various data processing operations executed at the third data processing stage 202C are rendered on the GUI (i.e. the visual interface) for further analysis, and also the data processing is performed based on the preset settings, knowledgebase, and panels selected and applied via the rendered GUI. Thus, in addition to first preset setting 250A and second preset setting 250B, a third preset setting 250C is provided and is selectable via the GUI. The third preset 250C setting is panel-agnostic and is used for configurating a report template that is used for decision support for the assessment of a disease(s). Other research preset options 250D are also provided and selectable for visual analysis. A fourth preset setting 250E is selectable via the GUI that allows cohort analysis and filtering to be performed based on shared alleles detected in different steps. A fifth preset setting 250F is selectable via the GUI that allows STR, NTR, SNP linkage analysis on multiple pedigrees to be executed concurrently based on shared alleles detected in different steps and visualized via the GUI in the sequence alignment.

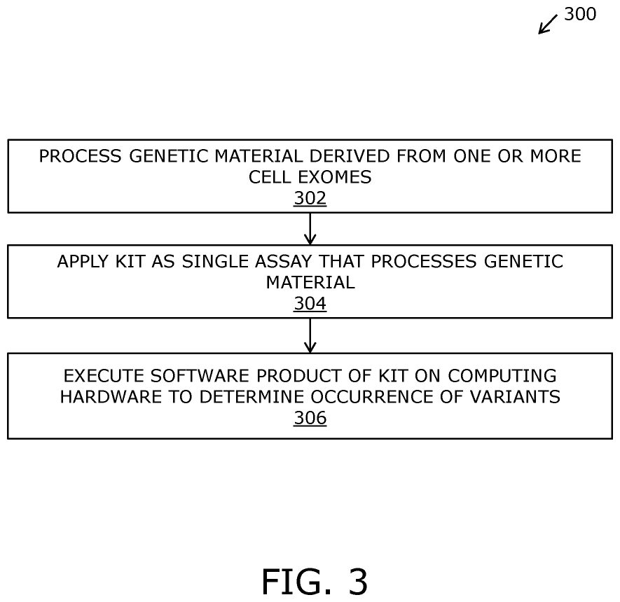

Referring to FIG. 3, there is shown a flowchart 300 depicting steps of a method of using a kit that performs a wet-lab assay, in accordance with an embodiment of the present disclosure. The method is implemented using a kit. The kit, when in use, performs a wet-lab assay. As shown, at a step 302, the assay processes genetic material that is derived from one or more cell exomes, wherein the assay detects single nucleotide variants (SNVs), indels and copy number variations (CNVs) in genetic DNA readout from the genetic material. At a step 304, the kit is applied as a single assay that processes the genetic material. At a step 306, the software product of the kit is executed on the computing hardware to cause the computing hardware to invoke one or more algorithms to process the genetic DNA readout by comparing portions of the genetic DNA readout against one or more DNA sequence transcripts, to determine an occurrence of variants corresponding to the one or more DNA sequence transcripts in the DNA readout data. Moreover, at the step 306, an algorithm is configured to detect both SNVs and CNVs in the genetic DNA readout from the genetic material. Further, an algorithm is configured to annotate clinically relevant CNVs present in the genetic DNA readout from the genetic material. Furthermore, an algorithm is configured to prioritize one or more portions of the genetic DNA readout from the genetic material depending on phenotype associated with the one or more portions. Moreover, the algorithm is configured to detect variant calling for pharmacogenomic (PGx) markers and sample tracking SNPs.

The steps 302, 304, and 306 are only illustrative and other alternatives can also be provided where one or more steps are added, one or more steps are removed, or one or more steps are provided in a different sequence without departing from the scope of the claims herein.

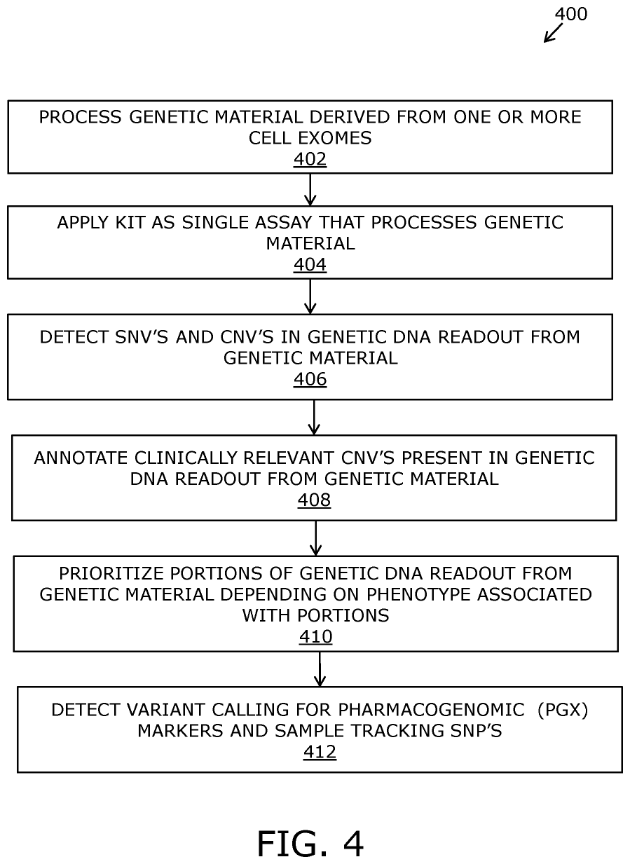

Referring to FIG. 4, there is shown a flowchart 400 depicting steps of a method of using a kit that performs a wet-lab assay, in accordance with another embodiment of the present disclosure. As shown, at a step 402, genetic material that is derived from a cell exome of a subject is processed. At a step 404, a kit, when in use with the apparatus, is applied as a single assay for processing the genetic material derived above step. At a step 406, the SNVs and the CNVs are detected in the genetic DNA readout from the genetic material. At a step 408, clinically relevant CNVS that are present in the genetic DNA readout of the genetic material are annotated. At a step 410, portions of the genetic DNA readout are prioritized from the genetic material depending upon a phenotype associated with the portions of the genetic DNA readout. At a step 412, variant calling for pharmacogenomic (PGx) markers and separately sample tracking SNPs are detected.

The steps 402, 404, 406, 408, 410 and 412 are only illustrative and other alternatives can also be provided where one or more steps are added, one or more steps are removed, or one or more steps are provided in a different sequence without departing from the scope of the claims herein.

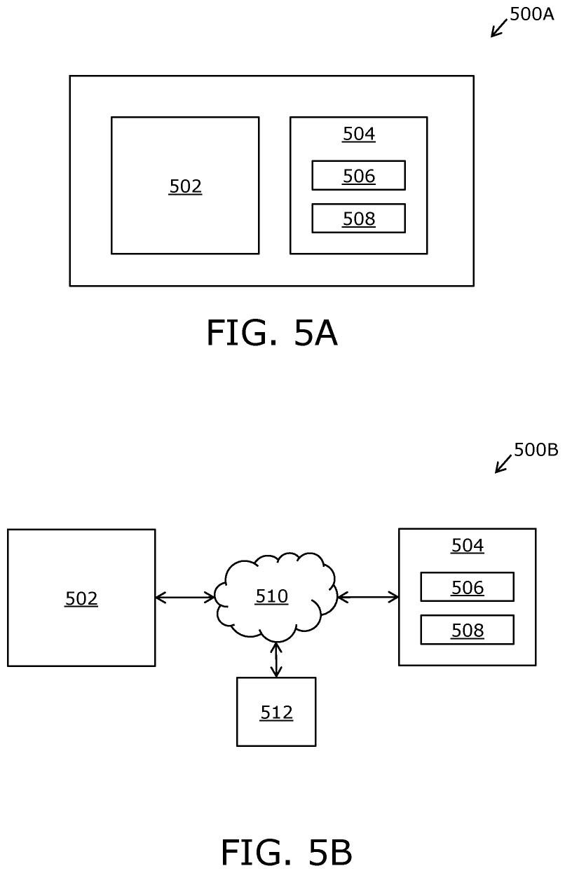

Referring to FIG. 5A, there is shown a block diagram of a system 500A that acquires and processes genomic sequence dataset to detect copy number variants (CNVs), in accordance with an embodiment of the present disclosure. As shown, the system 500A comprise an apparatus 502 and a computing arrangement 504. The apparatus 502 is configured to process at least a portion of a genome of a subject to generate a raw genomic sequence dataset. Moreover, the computing arrangement 504 comprises a data memory device 506 and a control circuitry 508. The control circuitry 508 is configured to acquire the raw genomic sequence dataset from the apparatus 502 as well as a plurality of candidate CNV detection applications prestored in the data memory device 506. Moreover, the control circuitry 508 is configured to execute a first CNV calling to obtain baseline CNVs in randomly selected regions of the raw genomic sequence dataset by use of each of the plurality of candidate CNV detection applications. Notably, the baseline CNVs are pre-existent CNVs in the raw genomic sequence dataset recognised as a ground truth. Furthermore, the control circuitry 508 is configured to combine the baseline CNVs obtained from each of the plurality of candidate CNV detection applications to generate a set of baseline CNVs. Moreover, the control circuitry 508 is configured to generate a simulated genomic sequence dataset by simulation of a set of artificial CNVs in at least one target region of the raw genomic sequence dataset by use of a simulation application (e.g. a zimmer tool) prestored in the data memory device 506. Notably, the simulated genomic sequence dataset comprises the set of artificial CNVs and the set of baseline CNVs. Furthermore, the control circuitry 508 is configured to record a location of each artificial CNV of the set of artificial CNVs and each baseline CNV of the set of baseline CNVs in the simulated genomic sequence dataset. Moreover, the control circuitry 508 is configured to execute a second CNV calling in the simulated genomic sequence dataset by use of each of the plurality of candidate CNV detection applications. Furthermore, the control circuitry 508 is configured to eliminate the set of baseline CNVs from CNVs obtained from the second CNV calling in the simulated genomic sequence dataset to obtain a set of new CNVs. Moreover, the control circuitry 508 is configured to determine a location of each new CNV of the set of new CNVs in the simulated genomic sequence dataset based on the recorded location of the set of artificial CNVs. Furthermore, the control circuitry 508 is configured to determine a degree of recall and a degree of precision associated with each of the plurality of candidate CNV detection applications based on a comparison of the location of the set of new CNVs with the location of the set of artificial CNVs. Moreover, the control circuitry 508 is configured to select one of the plurality of candidate CNV detection applications as being optimal, based on a combination of the degree of recall and the degree of precision for calling the copy number variants in genomic sequence data. Furthermore, the control circuitry 508 is configured to utilise the selected candidate CNV detection application for calling of CNVs in the genomic sequence data.

Referring to FIG. 5B, there is shown an illustration of a network environment of a system 500B that acquires and processes genomic sequence dataset to detect one or more copy number variants (CNVs), in accordance with another embodiment of the present disclosure. FIG. 5B is described in conjunction with elements from FIG. 5A. As shown, in the system 500B, the apparatus 502 and the computing arrangement 504 are communicatively coupled via a data communication network 510. The computing arrangement 504 comprises the data memory device 506 and the control circuitry 508. The data communication network 510 is a wired or wireless communication network. Further shown, is a wet-laboratory arrangement 512 that is communicatively coupled to the computing arrangement 504 and to the apparatus 502. The wet-laboratory arrangement 512 is configured process a biological sample of the subject to derive at least the portion of the genome of the subject to generate the raw genomic sequence dataset.

It will be appreciated by a person skilled in the art that the FIGS. 1A and 1B include a simplified illustration of the system 500A and 500B for the sake of clarity only, which should not unduly limit the scope of the claims herein. The person skilled in the art will recognize many variations, alternatives, and modifications of embodiments of the present disclosure.

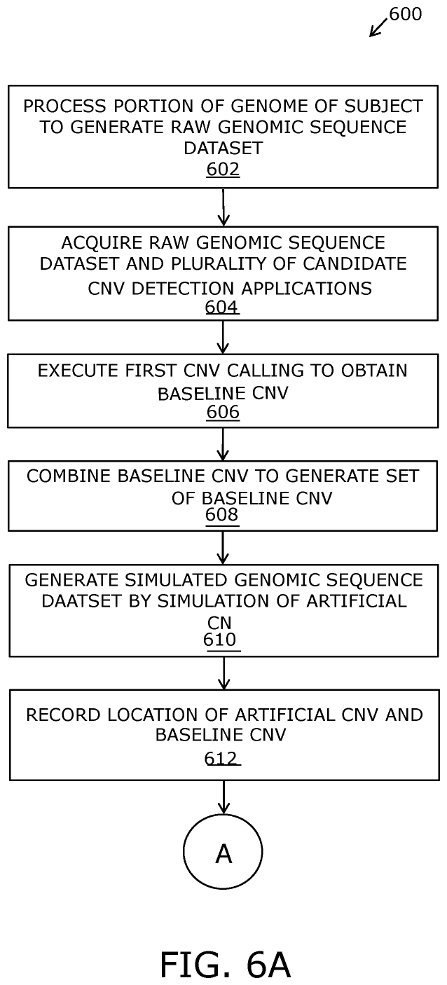

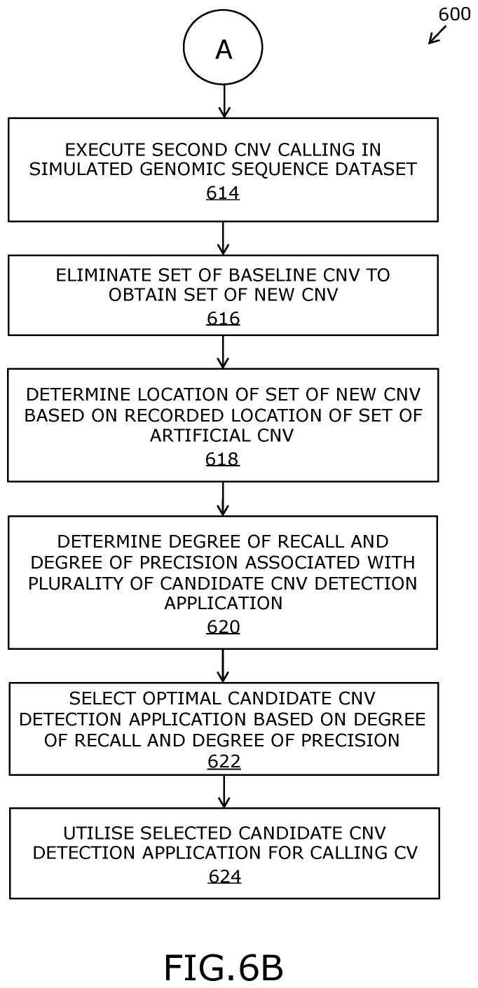

Referring to FIGS. 6A and 6B, there is shown a flowchart 600 depicting steps of a method for (of) acquiring and processing genomic sequence dataset to detect one or more copy number variants (CNVs), in accordance with an embodiment of the present disclosure. The method is implemented using a system that comprises an apparatus and a computing arrangement.

At a step 602, at least a portion of a genome of a subject is processed to generate a raw genomic sequence dataset, by use of the apparatus. At a step 604, the raw genomic sequence dataset from the apparatus and a plurality of candidate CNV detection applications prestored in a data memory device of the computing arrangement are acquired by use of a control circuitry of the computing arrangement. At a step 606, a first CNV calling is executed to obtain baseline CNVs in randomly selected regions of the raw genomic sequence dataset by use of each of the plurality of candidate CNV detection applications. Moreover, the baseline CNVs are pre-existent CNVs in the raw genomic sequence dataset recognized as a ground truth. At a step 608, the baseline CNVs obtained from each of the plurality of candidate CNV detection applications are combined to generate a set of baseline CNVs, by use of the control circuitry. At a step 610, a simulated genomic sequence dataset is generated by simulation of a set of artificial CNVs in at least one target region of the raw genomic sequence dataset by use of a simulation application prestored in the data memory device. Notably, the simulated genomic sequence dataset comprises the set of artificial CNVs and the set of baseline CNVs. At a step 612, a location of each artificial CNV of the set of artificial CNVs and each baseline CNV of the set of baseline CNVs in the simulated genomic sequence dataset is recorded. At a step 614, a second CNV calling is executed in the simulated genomic sequence dataset by use of each of the plurality of candidate CNV detection applications. At a step 616, the set of baseline CNVs is eliminated from CNVs obtained from the second CNV calling in the simulated genomic sequence dataset to obtain a set of new CNVs. At a step 618, a location of each new CNV of the set of new CNVs in the simulated genomic sequence dataset is determined based on the recorded location of the set of artificial CNVs. At a step 620, a degree of recall and a degree of precision associated with each of the plurality of candidate CNV detection applications is determined based on a comparison of the location of the set of new CNVs with the location of the set of artificial CNVs. At a step 622, one of the plurality of candidate CNV detection applications is selected as being optimal, based on a combination of the degree of recall and the degree of precision for calling the copy number variants in genomic sequence data. At a step 624, the selected candidate CNV detection application is utilized for calling of CNVs in the genomic sequence data by use of the control circuitry.

The steps 602, 604, 606, 608, 610, 612, 614, 616, 618, 620, 622 and 624 are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A kit for use in an apparatus for a genetic screening, wherein the kit, when in operation, performs a wet-lab assay, wherein the assay includes processing genetic material that is derived from one or more cell exomes, wherein the assay detects single nucleotide variants (SNVs), indels and copy number variations (CNVs) in genetic DNA readout from the genetic material,
characterized in that
the kit is executable as a single assay that processes the genetic material; and
the kit includes a software product that is executable on a computing hardware to cause the computing hardware to invoke one or more algorithms to process the genetic DNA readout by comparing portions of the genetic DNA readout against one or more DNA sequence transcripts, to determine an occurrence of variants corresponding to the one or more DNA sequence transcripts in the DNA readout data,
wherein the one or more algorithms include:
(i) an algorithm for detecting SNVs, indels and CNVs concurrently in the genetic DNA readout from the genetic material in the single assay;
(ii) an algorithm for annotating clinically relevant CNVs present in the genetic DNA readout from the genetic material;
(iii) an algorithm that prioritizes one or more portions of the genetic DNA readout from the genetic material depending on phenotype associated with the one or more portions;
(iv) an algorithm that detects variant calling for pharmacogenomic (PGx) markers;
(v) an algorithm configured to sample tracking SNPs in the single assay.

2. The kit of claim 1, characterized in that the software product includes an algorithm that, when executed on the computing hardware, provides a visualization arrangement implemented using a graphical user interface (GUI) to communicate visually results of detection in (i) to (iv).

3. The kit of claim 1, characterized in that the software product includes an algorithm that, when executed on the computing hardware, detects at least one of duplications and deletions in the DNA readout data relative of the DNA sequence transcripts, and wherein the genetic screening for which the kit is used includes at least one of a preconception screening, a preimplantation genetic screening, or an application related to assisted reproduction technology, and wherein the genetic material is processed using single cell sequencing.

4. The kit of claim 1, characterized in that the software product includes an algorithm that, when executed on the computing hardware, detects one or more intergenic variants present in the DNA readout data relative of the DNA sequence transcripts.

5. The kit of claim 1, characterized in that the software product includes algorithm that, when executed on the computing hardware, provides a combined SNV and CNV filtering and interpretation by a mode of genetic inheritance, wherein the mode of genetic inheritance includes a potential for recessive genes being present.

6. The kit of claim 1, characterized in that the one or more DNA sequence transcripts include consensus coding sequence (CCDS) transcripts.

7. The kit of claim 1, characterized in that the one or more DNA sequence transcripts include at least one morbid gene RefSeq transcript.

8. The kit of claim 7, characterized in that the one or more DNA sequence transcripts include at least 4091 morbid gene RefSeq transcripts.

9. The kit of claim 1, characterized in that the one or more DNA sequence transcripts include at least one fetal anomaly gene transcript.

10. The kit of claim 9, characterized in that the one or more DNA sequence transcripts include at least 2598 fetal anomalies gene transcripts.

11. The kit of claim 1, characterized in that the one or more DNA sequence transcripts include at least one epilepsy anomaly gene transcript.

12. The kit of claim 11, characterized in that the one or more DNA sequence transcripts include at least 5019 epilepsy gene Havana transcript features.

13. The kit of claim 1, characterized in that the one or more DNA sequence transcripts include at least one ACMG59 gene RefSeq transcript.

14. The kit of claim 1, characterized in that the one or more DNA sequence transcripts include likely pathogenic variants and non-coding variants of DNA sequence (ClinVar).

15. The kit of claim 1, characterized in that the one or more DNA sequence transcripts include at least one sample tracking SNV.

16. A method of using the kit of claim 1, wherein the kit, when in use, performs a wet-lab assay, wherein the assay includes processing genetic material that is derived from one or more cell exomes, wherein the assay detects single nucleotide variants (SNVs), indels and copy number variations (CNVs) in genetic DNA readout from the genetic material, characterized in that the method includes:
(i) applying the kit as a single assay that processes the genetic material; and
(ii) executing a software product of the kit on computing hardware to cause the computing hardware to invoke one or more algorithms to process the genetic DNA readout by comparing portions of the genetic DNA readout against one or more DNA sequence transcripts, to determine an occurrence of variants corresponding to the one or more DNA sequence transcripts in the DNA readout data,
wherein the one or more algorithms include:
(a) an algorithm for detecting SNVs, indels and CNVs concurrently in the genetic DNA readout from the genetic material in the single assay;
(b) an algorithm for annotating clinically relevant CNVs present in the genetic DNA readout from the genetic material;
(c) an algorithm that prioritizes one or more portions of the genetic DNA readout from the genetic material depending on phenotype associated with the one or more portions;
(d) an algorithm that detects variant calling for pharmacogenomic (PGx) markers; and
(e) an algorithm configured to sample tracking SNVs.

17. The method of claim 16, characterized in that the method is used to
implement the assay in a plurality of stages, wherein in a first selection stage of the plurality of stages, the method allows selecting a set of features-of-interest from a plurality of features that are configurable using the kit, wherein the plurality of features include exome sequencing preferences and a plurality of custom variants identification modules.

18. The method of claim 17, characterized in that the method is used to implement the assay in the plurality of stages, wherein in a second wet-lab stage of the plurality of stages, the method allows processing of the genetic material using the kit in accordance to the selected set of features-of-interest in the first selection stage to obtain the genetic DNA readout data from the genetic material, wherein the genetic DNA readout data corresponds to sequencing data, and wherein the kit is used in at least one of a preconception screening, a preimplantation genetic screening, or an application related to assisted reproduction technology, and wherein the genetic material is processed using single cell sequencing.

19. A method of claim 17, characterized in that the method is used to implement the assay in the plurality of stages, wherein in a third data processing pipeline stage of the plurality of stages, the method allows determination of the occurrence of variants in the DNA readout data in accordance to the selected set of features-of-interest in the first selection stage, wherein the determination of the occurrence of variants in the DNA readout data further comprises:
triggering a specific processing pipeline in accordance to the selected set of features-of-interest in the first selection stage;
executing unique molecular identifier (UMI) demultiplexing on the genetic DNA readout data;
executing mitochondrial (mtDNA) pipeline to measure heteroplasmic variants in the genetic DNA readout data;
detecting short tandem repeats (STR) and VNTR (variable number tandem repeats) in the genetic DNA readout data;
detecting mosaic variants in the genetic DNA readout data;
executing tagging of detected variants that meet gene mode of inheritance (MOI) with expected MOI in a family;
determining whether a detected variant is an inherited variant or a de novo variant; and
auto populating an evidence code when the detected variants match with prestored variant sequences acquired from a specified data source that defines gene variations and corresponding disorders.

20. The method of claim 16, characterized in that the method is used to implement the assay in a plurality of stages, wherein in a fourth visualization stage of the plurality of stages, the method allows rendering of a graphical user interface to communicate and interact with results of detection in the third data processing pipeline stage based on a plurality of defined settings.

21. The method of claim 16, wherein said processing genetic material comprises one, more or all of the following:
(a) extracting said genetic material from a sample taken from a subject;
(b) assessing purity of the extracted genetic material, preferably by measuring UV absorbance thereof;
(c) in case of said genetic material being RNA, reverse transcribing said RNA to obtain cDNA;
(d) in case of said genetic material being DNA or cDNA, shearing or digesting said genetic material to obtain fragments;
(e) enriching protein-coding regions, preferably by hybridizing to complementary oligonucleotides; and
(f) ligating the fragments obtained in (d) to adapters and annealing the ligation products to a solid carrier such as a glass slide.

22. The method of claim 21, wherein said sample is selected from tissue, biopsy, sample of a fetus, and a bodily fluid, said bodily fluid preferably being blood, throat swab, sputum, surgical drain fluid or amniotic fluid.

23. The method of claim 21, wherein said genetic material is DNA or RNA, preferably DNA.

24. A system that acquires and processes genomic sequence dataset to detect one or more copy number variants (CNVs) therein, the system comprising:
an apparatus configured to process at least a portion of a genome of a subject to generate a raw genomic sequence dataset; and
a computing arrangement comprising a data memory device and control circuitry, wherein the control circuitry is configured to:
acquire the raw genomic sequence dataset from the apparatus and a plurality of candidate CNV detection applications prestored in the data memory device;
execute a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth;
combine the baseline CNVs obtained from each of the plurality of candidate CNV detection applications to generate a set of baseline CNVs;
generate a simulated genomic sequence dataset by simulation of a set of artificial CNVs in at least one target region of the raw genomic sequence dataset by use of a simulation application prestored in the data memory device, wherein the simulated genomic sequence dataset comprises the set of artificial CNVs and the set of baseline CNVs;
record a location of each artificial CNV of the set of artificial CNVs and each baseline CNV of the set of baseline CNVs in the simulated genomic sequence dataset;
execute a second CNV calling in the simulated genomic sequence dataset by use of each of the plurality of candidate CNV detection applications;
eliminate the set of baseline CNVs from CNVs obtained from the second CNV calling in the simulated genomic sequence dataset to obtain a set of new CNVs;
determine a location of each new CNV of the set of new CNVs in the simulated genomic sequence dataset based on the recorded location of the set of artificial CNVs;
determine a degree of recall and a degree of precision associated with each of the plurality of candidate CNV detection applications based on a comparison of the location of the set of new CNVs with the location of the set of artificial CNVs;
select one of the plurality of candidate CNV detection applications as being optimal, based on a combination of the degree of recall and the degree of precision for calling the copy number variants in genomic sequence data; and
utilize the selected candidate CNV detection application for calling of CNVs in the genomic sequence data.

25. The system according to claim 24, wherein the control circuitry is further configured to determine the degree of recall associated with each of the plurality of candidate CNV detection applications by identification of:
a true positive, if a location of a new CNV of the set of new CNVs and a corresponding location of an artificial CNV of the set of artificial CNVs match;
a false positive, if a location of a new CNV of the set of new CNVs is detected at a location that is different than a location of an artificial CNV of the set of artificial CNVs; and
a false negative, if no new CNV of the set of new CNVs is detected at a location an artificial CNV of the set of artificial CNVs.

26. The system according to claim 24, wherein the control circuitry is further configured to measure an extent of overlap of a location of a new CNV of the set of new CNVs with a corresponding location of an artificial CNV of the set of artificial CNVs, for determination of the degree of precision associated with each of the plurality of candidate CNV detection applications.

27. The system according to claim 25, wherein the control circuitry is configured to allocate a highest degree of precision to a first candidate CNV detection application among the plurality of candidate CNV detection applications, based on the measured extent of overlap of the location of the new CNV of the set of new CNVs with the corresponding location of the artificial CNV of the set of artificial CNVs, by use of each of the plurality of candidate CNV detection applications.

28. The system according to claim 25, wherein the control circuitry is further configured to set a specified threshold for determination of the extent of overlap of the location of the new CNV of the set of new CNVs with the corresponding location of the artificial CNV of the set of artificial CNVs.

29. The system according to claim 24, wherein the apparatus is configured to perform at least one of: a whole genome sequencing, an exome sequencing to generate the raw genomic sequence dataset.

30. The system according to claim 24, wherein the control circuitry is further configured to generate a precision-recall curve relationship associated with each of the plurality of candidate CNV detection applications, and wherein the selection of one of the plurality of candidate CNV detection applications as optimal depends upon a balance between the degree of recall and the degree of precision, wherein the balance between the degree of recall and the degree of precision related to each of the plurality of candidate CNV detection applications is indicated by a corresponding area-under-precision-recall-curve in the generated precision-recall curve relationship.

31. The system according to claim 24, wherein the system further comprises a wet-laboratory arrangement, and wherein the wet-laboratory arrangement is configured to process a biological sample of the subject in the wet-laboratory arrangement to derive at least the portion of the genome of the subject to generate the raw genomic sequence dataset.

32. A system that processes a raw genomic sequence dataset to detect one or more copy number variants (CNVs) therein, the system comprising:
a computing arrangement comprising a data memory device and control circuitry, wherein the control circuitry is configured to:
acquire the raw genomic sequence dataset and a plurality of candidate CNV detection applications prestored in the data memory device;
execute a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth;
combine the baseline CNVs obtained from each of the plurality of candidate CNV detection applications to generate a set of baseline CNVs;
generate a simulated genomic sequence dataset by simulation of a set of artificial CNVs in at least one target region of the raw genomic sequence dataset by use of a simulation application prestored in the data memory device, wherein the simulated genomic sequence dataset comprises the set of artificial CNVs and the set of baseline CNVs;
record a location of each artificial CNV of the set of artificial CNVs and each baseline CNV of the set of baseline CNVs in the simulated genomic sequence dataset;
execute a second CNV calling in the simulated genomic sequence dataset by use of each of the plurality of candidate CNV detection applications;
eliminate the set of baseline CNVs from CNVs obtained from the second CNV calling in the simulated genomic sequence dataset to obtain a set of new CNVs;
determine a location of each new CNV of the set of new CNVs in the simulated genomic sequence dataset based on the recorded location of the set of artificial CNVs;
determine a degree of recall and a degree of precision associated with each of the plurality of candidate CNV detection applications based on a comparison of the location of the set of new CNVs with the location of the set of artificial CNVs;
select one of the plurality of candidate CNV detection applications as being optimal, based on a combination of the degree of recall and the degree of precision for calling the copy number variants in genomic sequence data; and
utilize the selected candidate CNV detection application for calling of CNVs in the genomic sequence data.

33. A method for acquiring and processing genomic sequence dataset to detect one or more copy number variants (CNVs) therein, wherein the method is implemented using a system that comprises an apparatus and a computing arrangement, wherein the method comprises:
processing, by use of the apparatus, at least a portion of a genome of a subject to generate a raw genomic sequence dataset;
acquiring, by use of a control circuitry of the computing arrangement, the raw genomic sequence dataset from the apparatus and a plurality of candidate CNV detection applications prestored in a data memory device of the computing arrangement;
executing, by use of the control circuitry, a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth;
combining, by use of the control circuitry, the baseline CNVs obtained from each of the plurality of candidate CNV detection applications to generate a set of baseline CNVs;
generating, by use of the control circuitry, a simulated genomic sequence dataset by simulation of a set of artificial CNVs in at least one target region of the raw genomic sequence dataset by use of a simulation application prestored in the data memory device, wherein the simulated genomic sequence dataset comprises the set of artificial CNVs and the set of baseline CNVs;
recording, by use of the control circuitry, a location of each artificial CNV of the set of artificial CNVs and each baseline CNV of the set of baseline CNVs in the simulated genomic sequence dataset;
executing, by use of the control circuitry, a second CNV calling in the simulated genomic sequence dataset by use of each of the plurality of candidate CNV detection applications;
eliminating, by use of the control circuitry, the set of baseline CNVs from CNVs obtained from the second CNV calling in the simulated genomic sequence dataset to obtain a set of new CNVs;
determining, by use of the control circuitry, a location of each new CNV of the set of new CNVs in the simulated genomic sequence dataset based on the recorded location of the set of artificial CNVs;
determining, by use of the control circuitry, a degree of recall and a degree of precision associated with each of the plurality of candidate CNV detection applications based on a comparison of the location of the set of new CNVs with the location of the set of artificial CNVs;
selecting, by use of the control circuitry, one of the plurality of candidate CNV detection applications as being optimal, based on a combination of the degree of recall and the degree of precision for calling the copy number variants in genomic sequence data; and
utilizing, by use of the control circuitry, the selected candidate CNV detection application for calling of CNVs in the genomic sequence data.

34. The method according to claim 33, wherein the method comprises determining, by the control circuitry, the degree of recall associated with each of the plurality of candidate CNV detection applications by identifying:
a true positive, if a location of a new CNV of the set of new CNVs matches with a corresponding location of an artificial CNV of the set of artificial CNVs;
a false positive, if a location of a new CNV of the set of new CNVs is detected at a location that is different than a location of an artificial CNV of the set of artificial CNVs; and
a false negative, if no new CNV of the set of new CNVs is detected at a location an artificial CNV of the set of artificial CNVs.

35. The method according to claim 33, wherein the method comprises measuring, by use of the control circuitry, an extent of overlap of a location of a new CNV of the set of new CNVs with a corresponding location of an artificial CNV of the set of artificial CNVs, for determination of the degree of precision associated with each of the plurality of candidate CNV detection applications.

36. The method according to claim 35, wherein the method further comprises allocating, by use of the control circuitry, a highest degree of precision to a first candidate CNV detection application among the plurality of candidate CNV detection applications, based on the measured extent of overlap of the location of the new CNV of the set of new CNVs with the corresponding location of the artificial CNV of the set of artificial CNVs, by use of each of the plurality of candidate CNV detection applications.

37. The method according to claim 35, wherein the method further comprises setting, by use of the control circuitry, a specified threshold for determination of the extent of overlap of the location of the new CNV of the set of new CNVs with the corresponding location of the artificial CNV of the set of artificial CNVs.

38. The method according to claim 33, wherein the method comprises generating, by use of the control circuitry, a precision-recall curve relationship associated with each of the plurality of candidate CNV detection applications, and wherein the selection of one of the plurality of candidate CNV detection applications as optimal depends upon a balance between the degree of recall and the degree of precision, wherein the balance between the degree of recall and the degree of precision related to each of the plurality of candidate CNV detection applications is indicated by a corresponding area-under-precision-recall-curve in the generated precision-recall curve relationship.

39. A computer program product comprising a non-transitory computer-readable storage medium having computer-readable instructions stored thereon, the computer-readable instructions being executable by a computerized device comprising processing hardware to execute a method as claimed in claim 33.

40. A method for acquiring and processing genomic sequence dataset to detect one or more copy number variants (CNVs) therein, wherein the method is implemented using a system that comprises a computing arrangement, wherein the method comprises:
acquiring, by use of a control circuitry of the computing arrangement, a raw genomic sequence dataset and a plurality of candidate CNV detection applications prestored in a data memory device of the computing arrangement;
executing, by use of the control circuitry, a first CNV calling to obtain baseline CNVs in randomly selected regions of the raw genomic sequence dataset by use of each of the plurality of candidate CNV detection applications, wherein the baseline CNVs are pre-existent CNVs in the raw genomic sequence dataset recognized as a ground truth;
combining, by use of the control circuitry, the baseline CNVs obtained from each of the plurality of candidate CNV detection applications to generate a set of baseline CNVs;
generating, by use of the control circuitry, a simulated genomic sequence dataset by simulation of a set of artificial CNVs in at least one target region of the raw genomic sequence dataset by use of a simulation application prestored in the data memory device, wherein the simulated genomic sequence dataset comprises the set of artificial CNVs and the set of baseline CNVs;
recording, by use of the control circuitry, a location of each artificial CNV of the set of artificial CNVs and each baseline CNV of the set of baseline CNVs in the simulated genomic sequence dataset;
executing, by use of the control circuitry, a second CNV calling in the simulated genomic sequence dataset by use of each of the plurality of candidate CNV detection applications;
eliminating, by use of the control circuitry, the set of baseline CNVs from CNVs obtained from the second CNV calling in the simulated genomic sequence dataset to obtain a set of new CNVs;
determining, by use of the control circuitry, a location of each new CNV of the set of new CNVs in the simulated genomic sequence dataset based on the recorded location of the set of artificial CNVs;
determining, by use of the control circuitry, a degree of recall and a degree of precision associated with each of the plurality of candidate CNV detection applications based on a comparison of the location of the set of new CNVs with the location of the set of artificial CNVs;
selecting, by use of the control circuitry, one of the plurality of candidate CNV detection applications as being optimal, based on a combination of the degree of recall and the degree of precision for calling the copy number variants in genomic sequence data; and
utilizing, by use of the control circuitry, the selected candidate CNV detection application for calling of CNVs in the genomic sequence data.

41. The kit of claim 1, wherein detection of the copy number variations (CNVs) in genetic DNA readout from the genetic material further comprises a control circuitry configured to:
receive the genetic DNA readout and a plurality of candidate CNV detection applications;
execute a first CNV calling to obtain baseline CNVs in randomly selected regions of the genetic DNA readout by use of each of the plurality of candidate CNV detection applications, wherein the baseline CNVs are pre-existent CNVs in the genetic DNA readout recognized as a ground truth;
combine the baseline CNVs obtained from each of the plurality of candidate CNV detection applications to generate a set of baseline CNVs;
generate a simulated genomic sequence dataset by simulation of a set of artificial CNVs in at least one target region of the genetic DNA readout by use of a simulation application, wherein the simulated genomic sequence dataset comprises the set of artificial CNVs and the set of baseline CNVs;
record a location of each artificial CNV of the set of artificial CNVs and each baseline CNV of the set of baseline CNVs in the simulated genomic sequence dataset;
execute a second CNV calling in the simulated genomic sequence dataset by use of each of the plurality of candidate CNV detection applications;
eliminate the set of baseline CNVs from CNVs obtained from the second CNV calling in the simulated genomic sequence dataset to obtain a set of new CNVs;
determine a location of each new CNV of the set of new CNVs in the simulated genomic sequence dataset based on the recorded location of the set of artificial CNVs;
determine a degree of recall and a degree of precision associated with each of the plurality of candidate CNV detection applications based on a comparison of the location of the set of new CNVs with the location of the set of artificial CNVs;
select one of the plurality of candidate CNV detection applications as being optimal, based on a combination of the degree of recall and the degree of precision for calling the copy number variants in genomic sequence data; and
utilize the selected candidate CNV detection application for calling of CNVs in the genomic sequence data.

42. The kit according to claim 41, wherein the control circuitry is further configured to determine the degree of recall associated with each of the plurality of candidate CNV detection applications by identification of:
a true positive, if a location of a new CNV of the set of new CNVs and a corresponding location of an artificial CNV of the set of artificial CNVs match;
a false positive, if a location of a new CNV of the set of new CNVs is detected at a location that is different than a location of an artificial CNV of the set of artificial CNVs; and
a false negative, if no new CNV of the set of new CNVs is detected at a location an artificial CNV of the set of artificial CNVs.

43. The kit according to claim 41, wherein the control circuitry is further configured to measure an extent of overlap of a location of a new CNV of the set of new CNVs with a corresponding location of an artificial CNV of the set of artificial CNVs, for determination of the degree of precision associated with each of the plurality of candidate CNV detection applications.

44. The kit according to claim 43, wherein the control circuitry is configured to allocate a highest degree of precision to a first candidate CNV detection application among the plurality of candidate CNV detection applications, based on the measured extent of overlap of the location of the new CNV of the set of new CNVs with the corresponding location of the artificial CNV of the set of artificial CNVs, by use of each of the plurality of candidate CNV detection applications.

45. The kit according to claim 43, wherein the control circuitry is further configured to set a specified threshold for determination of the extent of overlap of the location of the new CNV of the set of new CNVs with the corresponding location of the artificial CNV of the set of artificial CNVs.

46. The kit according to claim 41, wherein the genetic DNA readout is generated by whole genome sequencing, an exome sequencing, or both.

47. The kit according to claim 41, wherein the control circuitry is further configured to generate a precision-recall curve relationship associated with each of the plurality of candidate CNV detection applications, and wherein the selection of one of the plurality of candidate CNV detection applications as optimal depends upon a balance between the degree of recall and the degree of precision, wherein the balance between the degree of recall and the degree of precision related to each of the plurality of candidate CNV detection applications is indicated by a corresponding area-under-precision-recall-curve in the generated precision-recall curve relationship.

48. The kit according to claim 41, further comprising a wet-laboratory configured to process a biological sample of the subject in the wet-laboratory arrangement to derive at least the portion of the genome of the subject to generate the genetic DNA readout.
